SEQUENCE LISTING (27 sequences)

SEQ ID NO: 1 - protein, 119 aa, Artificial Sequence
  Heavy chain Consensus Sequence
  misc_feature (27)..(27): X is G or Y
  misc_feature (28)..(28): X is T or S
  misc_feature (30)..(30): X is S or T
  misc_feature (56)..(56): X is G or A
  misc_feature (67)..(67): X is R or K
  misc_feature (68)..(68): X is V or A
  misc_feature (70)..(70): X is I or L
  misc_feature (72)..(72): X is A or V
QVQLVQSGAEVKKPGSSVKVSCKASGXXFXGYTMNWVRQAPGQGLEWMGLITPYNXASSYNQKFRGXXTXTXDKSTSTAYMELSSLRSEDTAVYYCARGGYDGRGFDYWGQGTTVTVSS

SEQ ID NO: 2 - protein, 119 aa, Artificial Sequence
  VH1-e CDRgraft IgG1
QVQLVQSGAEVKKPGSSVKVSCKASGGTFSGYTMNWVRQAPGQGLEWMGLITPYNGASSYNQKFRGRVTITADKSTSTAYMELSSLRSEDTAVYYCARGGYDGRGFDYWGQGTTVTVSS

SEQ ID NO: 3 - protein, 119 aa, Artificial Sequence
  VH1- G27Y, T28S, S30T
QVQLVQSGAEVKKPGSSVKVSCKASGYSFTGYTMNWVRQAPGQGLEWMGLITPYNGASSYNQKFRGRVTITADKSTSTAYMELSSLRSEDTAVYYCARGGYDGRGFDYWGQGTTVTVSS

SEQ ID NO: 4 - protein, 119 aa, Artificial Sequence
  VH1-e R66K, V67A, I69L, A71V
QVQLVQSGAEVKKPGSSVKVSCKASGGTFSGYTMNWVRQAPGQGLEWMGLITPYNGASSYNQKFRGKATLTVDKSTSTAYMELSSLRSEDTAVYYCARGGYDGRGFDYWGQGTTVTVSS

SEQ ID NO: 5 - protein, 119 aa, Artificial Sequence
  VH1-e G27Y, T28S, S30T R66K, V67A, I69L, A71V
QVQLVQSGAEVKKPGSSVKVSCKASGYSFTGYTMNWVRQAPGQGLEWMGLITPYNGASSYNQKFRGKATLTVDKSTSTAYMELSSLRSEDTAVYYCARGGYDGRGFDYWGQGTTVTVSS

SEQ ID NO: 6 - protein, 119 aa, Artificial Sequence
  VH1-e G27Y, T28S, S30T, R66K, V67A, A71V
QVQLVQSGAEVKKPGSSVKVSCKASGYSFTGYTMNWVRQAPGQGLEWMGLITPYNGASSYNQKFRGKATITVDKSTSTAYMELSSLRSEDTAVYYCARGGYDGRGFDYWGQGTTVTVSS

SEQ ID NO: 7 - protein, 119 aa, Artificial Sequence
  VH1-e G27Y, R66K, V67A, A71V
QVQLVQSGAEVKKPGSSVKVSCKASGYTFSGYTMNWVRQAPGQGLEWMGLITPYNGASSYNQKFRGKATITVDKSTSTAYMELSSLRSEDTAVYYCARGGYDGRGFDYWGQGTTVTVSS

SEQ ID NO: 8 - protein, 119 aa, Artificial Sequence
  VH1-e G27Y, G55A, R66K, V67A, A71V
QVQLVQSGAEVKKPGSSVKVSCKASGYTFSGYTMNWVRQAPGQGLEWMGLITPYNAASSYNQKFRGKATITVDKSTSTAYMELSSLRSEDTAVYYCARGGYDGRGFDYWGQGTTVTVSS

SEQ ID NO: 9 - protein, 119 aa, Artificial Sequence
  VH1-e CDRgraft  G55A
QVQLVQSGAEVKKPGSSVKVSCKASGGTFSGYTMNWVRQAPGQGLEWMGLITPYNAASSYNQKFRGRVTITADKSTSTAYMELSSLRSEDTAVYYCARGGYDGRGFDYWGQGTTVTVSS

SEQ ID NO: 10 - protein, 106 aa, Artificial Sequence
  Light chain Consensus Sequence For VKIII and for CDRs
  misc_feature (1)..(1): X is E or D
  misc_feature (45)..(45): X is L or R
XIVLTQSPATLSLSPGERATLSCSASSSVSYMHWYQQKPGQAPRXLIYDTSKLASGIPARFSGSGSGTDFTLTISSLEPEDFAVYYCQQWSKHPLTFGGGTKVEIK

SEQ ID NO: 11 - protein, 106 aa, Artificial Sequence
  VKIII-L6 CDRgraft
EIVLTQSPATLSLSPGERATLSCSASSSVSYMHWYQQKPGQAPRLLIYDTSKLASGIPARFSGSGSGTDFTLTISSLEPEDFAVYYCQQWSKHPLTFGGGTKVEIK

SEQ ID NO: 12 - protein, 106 aa, Artificial Sequence
  VKIII-L6 E1D
DIVLTQSPATLSLSPGERATLSCSASSSVSYMHWYQQKPGQAPRLLIYDTSKLASGIPARFSGSGSGTDFTLTISSLEPEDFAVYYCQQWSKHPLTFGGGTKVEIK

SEQ ID NO: 13 - protein, 106 aa, Artificial Sequence
  VKIII-L6 L46R
EIVLTQSPATLSLSPGERATLSCSASSSVSYMHWYQQKPGQAPRRLIYDTSKLASGIPARFSGSGSGTDFTLTISSLEPEDFAVYYCQQWSKHPLTFGGGTKVEIK

SEQ ID NO: 14 - protein, 106 aa, Artificial Sequence
  VKIII-L6 E1D L46R
DIVLTQSPATLSLSPGERATLSCSASSSVSYMHWYQQKPGQAPRRLIYDTSKLASGIPARFSGSGSGTDFTLTISSLEPEDFAVYYCQQWSKHPLTFGGGTKVEIK

SEQ ID NO: 15 - protein, 106 aa, Artificial Sequence
  VKI-L12 CDRgraft
DIQMTQSPSTLSASVGDRVTITCSASSSVSYMHWYQQKPGKAPKLLIYDTSKLASGVPSRFSGSGSGTEFTLTISSLQPDDFATYYCQQWSKHPLTFGGGTKVEIK

SEQ ID NO: 16 - protein, 5 aa, Artificial Sequence
  CDR1 heavy chain
GYTMN

SEQ ID NO: 17 - protein, 17 aa, Artificial Sequence
  CDR2 heavy chain G55A
LITPYNAASSYNQKFRG

SEQ ID NO: 18 - protein, 17 aa, Artificial Sequence
  CDR2 heavy chain unmodified
LITPYNGASSYNQKFRG

SEQ ID NO: 19 - protein, 10 aa, Artificial Sequence
  CDR3 heavy chain
GGYDGRGFDY

SEQ ID NO: 20 - protein, 10 aa, Artificial Sequence
  CDR1 light chain
SASSSVSYMH

SEQ ID NO: 21 - protein, 7 aa, Artificial Sequence
  CDR2 light chain
DTSKLAS

SEQ ID NO: 22 - protein, 9 aa, Artificial Sequence
  CDR3 light chain
QQWSKHPLT

SEQ ID NO: 23 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 107 aa, Artificial Sequence [sequence omitted]

SEQ ID NO: 25 - protein, 119 aa, Artificial Sequence
  SS1 heavy chain variable region
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 26 - protein, 106 aa, Artificial Sequence
  SS1 light chain variable region
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 27 - protein, 119 aa, Artificial Sequence
  SS1 heavy chain variable region G55A
QVQLQQSGPELEKPGASVKISCKASGYSFTGYTMNWVKQSHGKSLEWIGLITPYNAASSYNQKFRGKATLTVDKSSSTAYMDLLSLTSEDSAVYFCARGGYDGRGFDYWGSGTPVTVSS

(19) United States
(12) Patent Application Publication
(10) Pub. No.: US 2022/0249685 A1
(43) Pub. Date: Aug. 11, 2022

(54) ANTI-MESOTHELIN ANTIBODIES AND IMMUNOCONJUGATES THEREOF

(71) Applicant: Silverback Therapeutics, Inc., Seattle, WA (US)

(72) Inventors: Brenda STEVENS, Seattle, WA (US); Peter Robert BAUM, Seattle, WA (US); Robert DUBOSE, Seattle, WA (US); Valerie ODEGARD, Seattle, WA (US)

(21) Appl. No.: 17/618,857

(22) PCT Filed: Jun. 18, 2020
(86) PCT No.: PCT/US2020/038365

§ 371 Date: Dec. 13, 2021

Related U.S. Application Data
(60) Provisional application No. 62/863,463, filed on Jun. 19, 2019.

(51) Int. Cl. A61K 47/68; A61P 35/00; C07K 16/28; C07K 16/30

(52) U.S. Cl. CPC A61K 47/6851; A61K 47/6849; A61K 47/6803; A61K 2039/545; C07K 16/2887; C07K 16/2803; C07K 16/30; A61P 35/00

(57) ABSTRACT

Anti-mesothelin antibodies and conjugates comprising such antibodies are disclosed herein as well as the use of such conjugates in the treatment of disease, such as cancer.

This application claims the benefit of priority of U.S. Provisional Application No. 62/863,463, filed Jun. 19, 2019, which is incorporated by reference herein in its entirety for any purpose.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

The mesothelin gene (MSLN) encodes a 71-kilodalton (kDa) precursor protein that is processed to a 40-kDa protein termed mesothelin, which is a glycosyl-phosphatidylinositol-anchored glycoprotein present on the cell surface (Chang, et al, Proc Natl Acad Sci USA (1996) 93:136-40). Mesothelin is a differentiation antigen whose expression in normal human tissues is limited to mesothelial cells lining the body cavity, such as the pleura, pericardium and peritoneum. Mesothelin is also highly expressed in several different human cancers, including mesotheliomas, pancreatic adenocarcinomas, ovarian cancers, stomach and lung adenocarcinomas. (Hassan, et al., Eur J Cancer (2008) 44:46-53)

Mesothelin is an appropriate target for methods of disease treatment and there is a need for effective immunoconjugates to target mesothelin. This invention addresses this and other needs.

### SUMMARY

The present invention provides, inter alia, anti-mesothelin antibodies that specifically bind to human mesothelin.

In some aspects, the anti-mesothelin antibodies are conjugated via a linker to a cytotoxic agent or an immune-stimulatory compound. In some embodiments, the linker is a cleavable linker. In other embodiments, the linker is a non-cleavable linker.

In some aspects, a conjugate of the present invention comprises an antibody that specifically binds to human mesothelin conjugated via a linker to a benzazepine compound. The benzazepine compound may be, for example, a compound of Formula (IA):

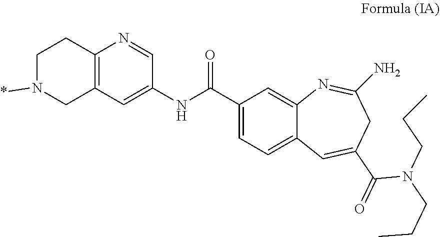 [SMILES: *N1CCc2ncc(NC(=O)c3ccc4c(c3)N=C(N)CC(C(=O)N(CCC)CCC)=C4)cc2C1]

or a salt thereof, wherein * indicates point of attachment to the linker. The linker can be, for example a cleavable or non-cleavable linker.

Also provided herein are methods for treating a mesothelin-expressing cancer and methods for eliciting targeted immune stimulation in a subject with a mesothelin-expressing cancer comprising administering to the subject a conjugate disclosed herein.

Pharmaceutical compositions comprising the conjugates described herein are also provided.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1 demonstrates that the anti-mesothelin antibodies are able to bind to a mesothelin expressing tumor cell line, Ovcar 3.

FIGS. 2A-2C demonstrate that the anti-mesothelin TLR8 agonist conjugates are able to bind to mesothelin expressing tumor cells with a similar EC₅₀ as the unconjugated anti-mesothelin antibodies and bind similarly to cynomolgus MSLN. Binding is to transfected cynomolgus MSLN cells (2A), OVCAR3 cells (2B) and NCI-N87 cells (2C).

FIGS. 3A-3B demonstrate that the anti-mesothelin TLR8 agonist conjugates are able to increase production of the pro-inflammatory cytokine, TNFα, by human PBMCs in the presence of HEK293 cells transfected with human MSLN (3A) but not in non-transfected HEK293 cells lacking MSLN expression (3B).

FIGS. 4A-4C demonstrate that the anti-mesothelin TLR8 agonist conjugates can increase production of TNFα by human PBMCs in the presence of various tumor cell lines expression mesothelin such as the NCI-N87 cell line (4A) and the Ovcar 3 cell line (4B), but not in non-MSLN expressing cell lines such as HEK-293 (4C).

FIGS. 5A-5B demonstrate that the anti-mesothelin TLR8 agonist conjugates are able to increase production of TNFα by cynomolgus PBMCs in the presence of HEK293 cells transfected with cynomolgus MSLN (5A) but not in non-transfected HEK293 cells lacking MSLN expression (5B).

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present disclosure provides anti-mesothelin binding domains as well as conjugates and pharmaceutical compositions comprising such binding domains for use in the treatment of disease or modulating an immune response.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, the term “antibody” refers to an immunoglobulin molecule that specifically binds to, or is immunologically reactive toward, a specific antigen. The term antibody includes, for example, polyclonal, monoclonal, single domain, genetically engineered antibodies, and antigen binding fragments thereof. An antibody can be, for example, murine, chimeric, humanized, a heteroconjugate, bispecific, diabody, triabody, or tetrabody. An antigen binding fragment can include, for example, a Fab, Fab′, F(ab′)₂, Fv, rIgG, scFv, V_{HH}, V_{NAR}, or nanobody.

As used herein, an “antigen” refers to an antigenic substance that can elicit an immune response in a host. An antigen can be a peptide, polypeptide, protein, polysaccharide, lipid, or glycolipid, which can be recognized by an antibody. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

As used herein, “MSLN” and “mesothelin” refer to any native MSLN that results from expression and processing of MSLN in a cell. The term includes MSLN from any vertebrate source, including mammals such as primates (e.g., humans and cynomolgus monkeys) and rodents (e.g., mice and rats), unless otherwise indicated. The term also includes naturally occurring variants of MSLN, e.g., splice variants or allelic variants. The amino acid sequence of an exemplary human MSLN precursor protein (with signal peptide, amino acids 1-36) is shown in UniProtKB Accession No. Q13421.

As used herein, a “tumor antigen” refers to an antigenic substance present on a cancer cell that can be recognized by an antibody and is preferentially present on a cancer cell as compared to normal (non-cancerous) cells.

As used herein, an “Fc domain” refers to a domain from an Fc portion of an antibody or a domain from a non-antibody molecule that can specifically bind to an Fc receptor, such as a Fcgamma receptor or an FcRn receptor.

As used herein, “recognize” with regard to antibody interactions can refer to the specific association or binding between an antibody and an antigen. Specific association or specific binding does not require that the antigen binding domain does not associate with or bind to any other antigen, but rather that it preferentially associates with or binds to the antigen, as compared to association with or binding to an unrelated antigen.

As used herein, “specifically binds” and the like refers to the specific association or specific binding between the antibody and the antigen, as compared with the interaction of the antibody with a different antigen (i.e., non-specific binding). In some embodiments, an antibody that recognizes or specifically binds to an antigen has a dissociation constant (KD) of <<100 nM, <10 nM, <1 nM, <0.1 nM, <0.01 nM, or <0.001 nM (e.g. 10⁻⁸M or less, e.g. from 10⁻⁸M to 10⁻¹³ M, e.g., from 10⁻⁹M to 10⁻¹³M).

As used herein, “conjugate” refers to an antibody that is linked, e.g., covalently linked, either directly or through a linker to an immune-stimulatory compound or cytotoxic compound.

As used herein, an “immune stimulatory compound” is a compound that directly or indirectly activates or stimulates an immune cell, such as a myeloid cell or an antigen presenting cell.

As used herein, the term “cytotoxic agent” as used herein refers to a substance that inhibits or prevents a cellular function and/or causes cell death or destruction. Cytotoxic agents include, but are not limited to, chemotherapeutic agents, growth inhibitory agents, toxins (e.g., protein toxins, enzymatically active toxins of bacterial, fungal, plant, or animal origin, or fragments thereof), or radioactive isotopes.

As used herein, an “immune-stimulatory conjugate” refers to a conjugate that activates or stimulates the immune system or a portion thereof, as determined by an in vitro or in vivo assay.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

As used herein, a “myeloid cell agonist” refers to a compound that activates or stimulates an immune response by a myeloid cell.

As used herein, the term “B-cell depleting agent” refers to an agent that, when administered to a subject, causes a reduction in the number of B cells in the subject. In some embodiments, a B-cell depleting agent binds a B cell surface molecule, such as, for example, CD20, CD22, or CD19. In some embodiments, a B-cell depleting agent inhibits a B cell survival factor, such as, for example, BLyS or APRIL. B-cell depleting agents include, but are not limited to, anti-CD20 antibodies, anti-CD19 antibodies, anti-CD22 antibodies, anti-BLyS antibodies, TACI-Ig, BR3-Fc, and anti-BR3 antibodies. Nonlimiting exemplary B-cell depleting agents include rituximab, ocrelizumab, ofatumumab, epratuzumab, MEDI-51 (anti-CD19 antibody), belimumab, BR3-Fc, AMG-623, and atacicept.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein. If not otherwise stated, an alkylene chain preferably has from 1 to 20 carbon atoms, more preferably from 1 to 10 carbon atoms.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., of the structure. It will be understood that “substitution” or “substituted with” includes the implicit proviso that such substitution is in accordance with permitted valence of the substituted atom and the substituent, and that the substitution results in a stable compound, Le., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E- form (or cis- or trans- form). Furthermore, some chemical entities may exist in various tautomeric forms. Unless otherwise specified, compounds described herein are intended to include all Z-, E- and tautomeric forms as well. A “tautomer” refers to a molecule wherein a proton shift from one atom of a molecule to another atom of the same molecule is possible. In circumstances where tautomerization is possible, a chemical equilibrium of the tautomers will exist.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Antibodies

Provided herein are, inter alia, antibodies, including humanized antibodies, comprising complementarity determining regions (CDRs) of the SS1 antibody. In exemplary embodiments, included are antibodies comprising CDR1, CDR2 and CDR3 of the light chain variable region of the SS1 antibody as well as CDR1, CDR2 and CDR3 of the heavy chain variable region of the SS1 antibody as well as antibodies having a modified heavy chain CDR2. The SS1 antibody is a chimeric monoclonal antibody IgG/K with high affinity and specificity for mesothelin. See Chowdhury and Pastan, Journal of Immunological Methods 231 (1999) 83-91) and Chowdhury et al., Proc. Natl. Acad. Sci., 95 (1998) 669-674, each of which is incorporated herein by reference and for all purposes.

Also provided herein are conjugates, including immune-stimulatory conjugates, comprising murine, chimeric, or humanized antibodies comprising CDR1, CDR2 and CDR3 of the light chain variable region of the SS1 antibody as well as CDR1, CDR2 and CDR3 of the heavy chain variable region of the SS1 antibody as well such antibodies with a modified heavy chain CDR2. In some aspects, antibodies having a modified heavy chain CDR2 are more stable than corresponding antibodies without the modified heavy chain CDR2.

In some embodiments, an antibody comprises two identical light protein chains (light chains) and two identical heavy protein chains (heavy chains), associated by precisely located disulfide linkages. In embodiments wherein antibodies are conjugated via one or more cysteines to an immune-stimulatory compound or cytotoxic agent, some or all of these linkages may be broken. The N-terminal regions of the light and heavy chains together can form the antigen recognition site of each antibody. Structurally, various functions of an antibody can be confined to discrete protein domains (i.e., regions). The sites that can recognize and can bind to antigen consist of three complementarity determining regions (CDRs) that can lie within the variable heavy chain regions and variable light chain regions at the N-terminal portions of the two heavy and two light chains. The framework and constant domains can provide the general framework of the antibody and may not be involved directly in binding the antibody to an antigen, but, in the case of the constant domains, can be involved in various effector functions, such as participation of the antibody in antibody-dependent cellular cytotoxicity (ADCC).

The domains of natural light chain variable regions and heavy chain variable regions can have the same general structures, and each domain can comprise four framework regions, whose sequences can be somewhat conserved, connected by three hyper-variable regions or CDRs. The four framework regions can largely adopt a (3-sheet conformation and the CDRs can form loops connecting, and in some aspects forming part of, the (3-sheet structure. The CDRs in each chain can be held in close proximity by the framework regions and, with the CDRs from the other chain, can contribute to the formation of the antigen binding site.

An antibody can comprise one or more light chain (LC) CDRs (LCDRs) and one or more heavy chain (HC) CDRs (HCDRs), one or more LCDRs or one or more HCDRs. For example, an antibody can comprise one or more of the following: a light chain complementary determining region 1 (LCDR1), a light chain complementary determining region 2 (LCDR2), or a light chain complementary determining region 3 (LCDR3). For another example, an antibody can comprise one or more of the following: a heavy chain complementary determining region 1 (HCDR1), a heavy chain complementary determining region 2 (HCDR2), or a heavy chain complementary determining region 3 (HCDR3). In some embodiments an antibody comprises all of the following: a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). Unless stated otherwise, the CDRs described herein can be defined according to Kabat.

An antibody may be of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. An antibody can further comprise a light chain and a heavy chain, often more than one chain. The heavy-chain constant regions (Fc) that corresponds to the different classes of immunoglobulins can be α, δ, ε, γ, and μ, respectively. The light chains can be one of either kappa (κ) or lambda (λ), based on the amino acid sequences of the constant domains. The Fc region may contain an Fc domain. An Fc receptor may bind an Fc domain.

In some embodiments, an antigen binding fragment (such as an antigen binding domain) of an antibody competes with the intact antibody for specific binding to the antigen. Antigen binding fragments include, for example, (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. In some embodiments, an antigen binding fragment (such as an antigen binding domain) comprises a heavy chain variable region and a light chain variable region.

F(ab′)₂ and Fab′ moieties may be produced by genetic engineering or by treating immunoglobulin (e.g., monoclonal antibody) with a protease such as pepsin and papain, and may include an antibody fragment generated by digesting immunoglobulin near the disulfide bonds existing between the hinge regions in each of the two H chains. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the three hypervariable regions of each variable domain may interact to define an antigen-binding site on the surface of the V_{H}-V_{L} dimer. A single variable domain (or half of an Fv comprising only three hypervariable regions specific for an antigen) may recognize and bind antigen, although the binding can be at a lower affinity than the affinity of the entire binding site.

An antibody may include an Fc region comprising an Fc domain. The Fc domain of an antibody may interact with FcRs found on immune cells. The Fc domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

A Fc domain may be modified to acquire or improve at least one constant region-mediated biological effector function relative to an unmodified antibody or Fc domain, e.g., to enhance FcγR interactions. An Fc domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface.

In some embodiments, an Fc domain of the antibody can exhibit increased binding affinity to one or more Fc receptors. In some embodiments, an Fc domain can exhibit increased binding affinity to one or more Fcgamma receptors. In some embodiments, an Fc domain can exhibit increased binding affinity to FcRn receptors. In some embodiments, an Fc domain can exhibit increased binding affinity to Fcgamma and FcRn receptors.

In some embodiments, an Fc domain of the antibody can exhibit reduced binding affinity to one or more Fc receptors. In some embodiments, an Fc domain can exhibit reduced binding affinity to one or more Fcgamma receptors. In some embodiments, an Fc domain can exhibit reduced binding affinity to FcRn receptors. In some embodiments, an Fc domain can exhibit reduced binding affinity to Fcgamma and FcRn receptors. In some embodiments, an Fc domain is an Fc null domain. In some embodiments, an Fc domain can exhibit reduced binding affinity to FcRn receptors, but have the same or increased binding affinity to one or more Fcgamma receptors as compared to a wildtype IgG. In some embodiments, an Fc domain can exhibit increased binding affinity to FcRn receptors, but have the same or decreased binding affinity to one or more Fcgamma receptors. As used herein, an “Fe null” refers to a domain that exhibits weak to no binding to any of the Fcgamma receptors. In some embodiments, an Fc null domain exhibits a reduction in binding affinity (e.g., increase in Kd) to Fc gamma receptors of at least 1000-fold.

The Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain to an Fc receptor, the Fc domain may comprise one or more amino acid substitutions that reduces the binding affinity of the Fc domain to an Fc receptor.

In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc domain can comprise a sequence of an IgG isoform that has been modified from the wild-type IgG sequence. In some embodiments, the Fc domain can comprise a sequence of the IgG1 isoform that has been modified from the wild-type IgG1 sequence. In some embodiments, the modification comprises substitution of one or more amino acids that reduce binding affinity of an IgG Fc domain to all Fcγ receptors. A modification can be substitution of E233, L234 and L235, such as E233P/L234V/L235A or E233P/L234V/L235A/AG236, according to the EU index of Kabat. A modification can be a substitution of P238, such as P238A; substitution of D265, such as D265A; substitution of N297, such as N297A; substitution of A327, such as A327Q; or substitution of P329, such as P239A, according to the EU index of Kabat.

In some embodiments, an IgG Fc domain comprises at least one amino acid substitution that reduces its binding affinity to FcγR1, as compared to a wild-type or reference IgG Fc domain. A modification can comprise a substitution at F241, such as F241A; substitution at F243, such as F243A; substitution at V264, such as V264A; or substitution at D265, such as D265A according to the EU index of Kabat.

In some embodiments, an IgG Fc domain comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain to FcγRII and FcγRIIIA receptors. A modification can be a substitution of D270, such as D270A; substitution of Q295, such as Q295A; or substitution of A327, such as A237S, according to the EU index of Kabat.

In some embodiments, the modification comprises substitution of one or more amino acids that increases binding affinity of an IgG Fc domain to FcγRII and FcγRIIIA receptors. A modification can be a substitution of T256, such as T256A; substitution of K290, such as K290A.

In some embodiments, the modification comprises substitution of one or more amino acids that increases binding affinity of an IgG Fc domain to FcγRII receptor. A modification can be a substitution of R255, such as R255A; substitution of E258, such as E258A; substitution of 5267, such as S267A; substitution of E272, such as E272A; substitution of N276, such as N276A; substitution of D280, such as D280A; substitution of H285, such as H285A; substitution of N286, such as N286A; substitution of T307, such as T307A; substitution of L309, such as L309A; substitution of N315, such as N315A; substitution of K326, such as K326A; substitution of P331, such as P331A; substitution of 5337, such as S337A; substitution of A378, such as A378A; or substitution of E430, such as E430, according to the EU index of Kabat.

In some embodiments, the modification comprises substitution of one or more amino acids that increases binding affinity of an IgG Fc domain to FcγRII receptor and reduces the binding affinity to FcγRIIIA receptor. A modification can be a substitution of H268, such as H268A; substitution of R301, such as R301A; or substitution of K322, such as K322A, according to the EU index of Kabat.

In some embodiments, the modification comprises substitution of one or more amino acids that decreases binding affinity of an IgG Fc domain to FcγRII receptor but does not affect the binding affinity to FcγRIIIA receptor. A modification can be a substitution of R292; or substitution of K414, such as K414A, according to the EU index of Kabat.

In some embodiments, the modification comprises substitution of one or more amino acids that decreases binding affinity of an IgG Fc domain to FcγRIIIA receptor. A modification can be substitution of F241 and F243, such as F241S/F243S or F241I/F2431, according to the EU index of Kabat.

In some embodiments, the modification comprises substitution of one or more amino acids that decreases binding affinity of an IgG Fc domain to FcγRIIIA receptor and does not affect the binding affinity to FcγRII receptor. A modification can be a substitution of 5239, such as S239A; substitution of E269, such as E269A; substitution of E293, such as E293A; substitution of Y296, such as Y296F; substitution of V303, such as V303A; substitution of A327, such as A327G; substitution of K338, such as K338A; or substitution of D376, such as D376A, according to the EU index of Kabat.

In some embodiments, the modification comprises substitution of one or more amino acids that increases binding affinity of an IgG Fc domain to FcγRIIIA receptor and does not affect the binding affinity to FcγRII receptor. A modification can be a substitution of E333, such as E333A; substitution of K334, such as K334A; substitution of A339, such as A339T; or substitution of S239 and 1332, such as S239D/I332E according to the EU index of Kabat.

In some embodiments, the modification comprises substitution of one or more amino acids that increases binding affinity of an IgG Fc domain to FcγRIIIA receptor. A modification can be substitution of L235, F243, R292, Y300 and P396, such as L235V/F243L/R292P/Y1300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

In some embodiments, the modification comprises substitution of one or more amino acids that increases binding affinity of an IgG Fc domain or region to FcγRII receptor and increases the binding affinity to FcγRIIIA receptor. A modification can be a substitution of S298, such as S298A; substitution of S239, 1332 and A330, such as S239D/I332E/A330L or substitution of S239 and 1332, such as S239D/I332E.

In some embodiments, the modification comprises substitution of one or more amino acids that increases binding affinity of an IgG Fc domain or region to a FcγRII receptor and decreases binding to a FcγRIII receptor such as L234A/L235A/G237A/K322A/S267E/L328F.

In some embodiments, the modification comprises substitution of one or more amino acids that increases binding affinity of an IgG Fc domain or region to a FcγRII receptor decreases binding to a FcγRIII receptor such as S267E/L328F.

Other substitutions in an IgG Fc domain that affect its interaction with one or more Fcγreceptors are disclosed in U.S. Patent Nos. 7,317,091 and 8,969,526 (the disclosures of which are incorporated by reference herein).

In some embodiments, an IgG Fc domain comprises at least one amino acid substitution that reduces the binding affinity to FcRn, as compared to a wild-type or reference IgG Fc domain. A modification can comprise a substitution at H435, such as H435A; a substitution at 1253, such as I253A; a substitution at H310, such as H310A or substitutions at 1253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P; such as M428L; substitution at N434, such as N434A; substitutions at T250 and M428, such as T250Q and M428L; substitutions at M428 and N434, such as M428L and N434S, N434A or N434H; substitutions at M252, S254 and T256, such as M252Y/S254T/T256E; or substitution of one or more amino acids selected from P257L, P257N, P257I, V279E, V279Q, V279Y, A281S, E283F, V284E, L306Y, T307V, V308F, Q311V, D376V, and N434H according to the EU index of Kabat. Other substitutions in an IgG Fc domain that affect its interaction with FcRn are disclosed in U.S. Pat. No. 9,803,023 (the disclosure of which is incorporated by reference herein).

Antibodies of the present invention can be humanized. Humanized forms of non-human (such as murine) antibodies can be full length immunoglobulins or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDR regions correspond to those of a non-human immunoglobulin and all or substantially all of the framework regions (FRs) are those of a human immunoglobulin sequence. The humanized antibody can also comprise at least a portion of an immunoglobulin constant region (Fc), typically that of a human immunoglobulin consensus sequence.

Antibodies of the present invention can be chimeric antibodies. Chimeric antibodies generally comprise non-human variable heavy and light chains (e.g., mice) and at least a portion of a human immunoglobulin constant region (Fc), typically that of a human immunoglobulin consensus sequence.

An antibody described herein may be a bispecific antibody or a dual variable domain antibody (DVD). Bispecific and DVD antibodies may be monoclonal, often human or humanized, antibodies that have binding specificities for at least two different antigens.

An antibody described herein may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

In certain embodiments, the antibody comprises a heavy chain variable region comprising complementarity determining regions (CDRs) having the amino acid sequences of the heavy chain variable region CDRs set forth in SEQ ID NO:1 and a light chain variable region comprising CDRs having the amino acid sequences of the light chain variable region CDRs set forth in SEQ ID NO:10. In some such aspects, the antibody comprises a heavy chain variable region comprising complementarity determining regions (CDRs) having the amino acid sequences of the heavy chain variable region CDRs set forth in SEQ ID NO:9. In other such aspects, the antibody comprises a heavy chain variable region comprising complementarity determining regions (CDRs) having the amino acid sequences of the heavy chain variable region CDRs set forth in SEQ ID NO:2. The CDRs can be identified, for example, via Kabat. For example, in embodiments wherein the CDRs are identified by Kabat, CDR 1 of the heavy chain is SEQ ID NO:16, CDR2 of the heavy chain is SEQ ID NO:17 or SEQ ID NO:18, CDR3 of the heavy chain is SEQ ID NO:19, CDR1 of the light chain is SEQ ID NO:20, CDR2 of the light chain is SEQ ID NO:21, and CDR3 of the light chain is SEQ ID NO:22. In some aspects, an antibody comprising a heavy chain CDR2 comprising the amino acid sequence of SEQ ID NO: 17 is more stable than an antibody comprising a heavy chain CDR2 comprising the amino acid sequence of SEQ ID NO: 18.

In some aspects, the antibody comprises a heavy chain variable region comprising the amino acid sequence set forth in SEQ ID NO:1. In some aspects, the antibody comprises a heavy chain variable region comprising the amino acid sequence set forth in SEQ ID NO:2. In some aspects, the antibody comprises a heavy chain variable region comprising the amino acid sequence set forth in SEQ ID NO:3. In some aspects, the antibody comprises a heavy chain variable region comprising the amino acid sequence set forth in SEQ ID NO:4. In some aspects, the antibody comprises a heavy chain variable region comprising the amino acid sequence set forth in SEQ ID NO:5. In some aspects, the antibody comprises a heavy chain variable region comprising the amino acid sequence set forth in SEQ ID NO:6. In some aspects, the antibody comprises a heavy chain variable region comprising the amino acid sequence set forth in SEQ ID NO:7. In some aspects, the antibody comprises a heavy chain variable region comprising the amino acid sequence set forth in SEQ ID NO:8. In some aspects, the antibody comprises a heavy chain variable region comprising the amino acid sequence set forth in SEQ ID NO:9.

In some aspects, the antibody comprises a light chain variable region comprising the amino acid sequence set forth in SEQ ID NO:10. In some aspects, the antibody comprises a light chain variable region comprising the amino acid sequence set forth in SEQ ID NO:11. In some aspects, the antibody comprises a light chain variable region comprising the amino acid sequence set forth in SEQ ID NO:12. In some aspects, the antibody comprises a light chain variable region comprising the amino acid sequence set forth in SEQ ID NO:13. In some aspects, the antibody comprises a light chain variable region comprising the amino acid sequence set forth in SEQ ID NO:14. In some aspects, the antibody comprises a light chain variable region comprising the amino acid sequence set forth in SEQ ID NO:15.

The antibodies of the present invention can comprise any combination of heavy chain variable region and light chain variable region as described herein. For example, the antibody can comprise a heavy chain variable region comprising the amino acid sequence set forth in SEQ ID Nos: 1, 2, 3, 4, 5, 6, 7, 8, or 9, and a light chain variable region comprising the amino acid sequence set forth in SEQ ID Nos: 10, 11, 12, 13, 14, or 15. For example, in exemplary embodiments, the antibody comprises a heavy chain variable region comprising the amino acid sequence set forth in SEQ ID NO:1 and a light chain variable region comprising the amino acid sequence set forth in SEQ ID NO:10 or SEQ ID NO:15; a heavy chain variable region comprising the amino acid sequence set forth in SEQ ID NO:8 and a light chain variable region comprising the amino acid sequence set forth in SEQ ID NO:11; a heavy chain variable region comprising the amino acid sequence set forth in SEQ ID NO:9 and a light chain variable region comprising the amino acid sequence set forth in SEQ ID NO:11; or a heavy chain variable region comprising the amino acid sequence set forth in SEQ ID NO:9 and a light chain variable region comprising the amino acid sequence set forth in SEQ ID NO:15.

In exemplary embodiments, the antibody comprises a heavy chain variable region comprising the amino acid sequence set forth in SEQ ID NO:25 or SEQ ID NO:27 and a light chain variable region comprising the amino acid sequence set forth in SEQ ID NO:26.

The antibodies can comprise any constant region known in the art. The light chain constant region can be, for example, a kappa- or lambda-type light chain constant region, The heavy chain constant region can be, for example, an alpha-, delta-, epsilon-, gamma-, or mu-type heavy chain constant regions. The light or heavy chain constant region can be a fragment, derivative, variant, or mutein of a naturally occurring constant region. The constant region can comprise an active Fc domain or a null Fc domain. In some aspects, the antibody comprises a wild-type IgG1 Fc domain or an IgG1 Fc domain variant having the same or substantially similar binding affinity to FcγRI, FcγRII, and FcγRIII as compared to a wild-type IgG1 Fc domain. In some aspects, the antibody comprises a wild-type IgG1 Fc domain or an IgG1 Fc domain variant having the same or substantially similar binding affinity to FcRn as compared to a wild-type IgG1 Fc domain. In other aspects, the antibody comprises a wild-type IgG1 Fc domain or an IgG1 Fc domain variant having increased or decreased affinity to one or more Fcγ receptors as compared to a wild-type IgG1 Fc domain. In some aspects, the antibodies may further comprise a heavy chain constant region comprising the amino acid sequence set forth in SEQ ID NO:23 and a light chain constant region comprising the amino acid sequence set forth in SEQ ID NO:24.

### Methods for Producing Anti-mesothelin Antibodies

Anti-mesothelin antibodies can be produced by any method known in the art for antibody production. As one example, an anti-mesothelin antibody can be produced by a method using an isolated nucleic acid sequence encoding an anti-mesothelin antibody, vectors and host cells comprising the nucleic acid sequence, and recombinant techniques for the production of the antibody. The nucleic acid sequence encoding the mesothelin antibody can be isolated into a replicable DNA vector for further cloning or for expression. DNA encoding an anti-mesothelin antibody can be readily isolated and sequenced using conventional procedures (e.g., by using oligonucleotide probes that are capable of binding specifically to genes encoding the heavy and light chains of the antibody). Many vectors known in the art can be used as a vector. The vector components generally can include, but are not limited to, one or more of the following: a signal sequence, an origin of replication, one or more marker genes, an enhancer element, a promoter, and a transcription-termination sequence. Suitable host cells for cloning or expressing the DNA vectors herein can be prokaryote, yeast, or higher eukaryote cells described herein. Suitable host cells for expression of glycosylated anti-mesothelin antibody can be derived from multicellular organisms. Examples of invertebrate cells can include, but are not limited to, plant and insect cells. Host cells used to produce an anti-mesothelin antibody can be cultured in a variety of commercial media. When using recombinant techniques, an anti-mesothelin antibody can be produced, for example, intracellularly, in the periplasmic space, or directly secreted into the medium. If the antibody is produced intracellularly, the particulate debris, either host cells or lysed fragments, can be removed, for example, by centrifugation or ultrafiltration. Where the antibody is secreted into the medium, supernatants from such expression systems can be concentrated using a commercially available protein concentration filter. A protease inhibitor such as phenylmethylsuphonyl fluoride can be included in any of the foregoing steps to inhibit proteolysis, and antibiotics can be included to prevent the growth of adventitious contaminants. The antibody composition prepared from the cells can be purified using, for example, hydroxylapatite chromatography, gel electrophoresis, dialysis, and affinity chromatography. The suitability of a protein A as an affinity ligand can depend on the species and isotype of any immunoglobulin Fc domain that may be present in the antibody. Other techniques for protein purification such as fractionation on the an ion-exchange column, ethanol precipitation, reverse-phase HPLC, chromatography on silica, chromatography on heparin SEPHAROSE™, chromatography on an anion- or cation-exchange resin (such as a polyaspartic acid column), chromatofocusing, SDS-PAGE, and ammonium-sulfate precipitation can also be used to recover the antibody. Following any preliminary purification step(s), the mixture comprising the anti-mesothelin antibody and contaminants can be subjected to low-pH hydrophobic-interaction chromatography. The methods for humanizing antibodies can include, for example, humanization uses CDR grafting (Jones et al., Nature 15 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., B W Metcalf, B J Dalton (Eds.) Cellular adhesion: molecular definition to therapeutic potential. Plenum Press, New York; 1994:291-312). Superhumanization (Tan, et al., 2002 J Immunol 169: 1119-25) is another variant humanization method that can be used to graft non-human CDRs into human germline antibody sequences having similar CDR canonical structures.

### Conjugates

A conjugate as described herein comprises an antibody and at least one linker attached to at least one drug. The drug can be, for example, an immune-stimulatory compound, such as, for example, a myeloid cell agonist or other agonist (e.g., TLR8 agonist, TLR7 agonist). Alternatively, the drug can be a cytotoxic agent. In some aspects, the present disclosure provides a conjugate represented by Formula II:

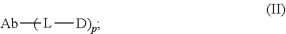

wherein:
Ab is the anti-mesothelin antibody,
L is the linker;
D is immune-stimulatory compound or cytotoxic agent;
p is selected from 1 to 20.

In a conjugate, the drug loading is represented by p, the number of drug-linker molecules per antibody. Depending on the context, p can represent the average number of drug-linker molecules per antibody, also referred to the average drug loading. In various embodiments, p can range from 1 to 20. In some conjugates, p is preferably from 1 to 8. In some preferred embodiments, when p represents the average drug loading, p ranges from about 2 to about 5. In some embodiments, p is about 2, about 3, about 4, or about 5 or about 8. The average drug-linker molecules per antibody in a preparation of conjugate may be characterized by conventional means such as mass spectroscopy, liquid chromatography/mass spectrometry (LC/MS), HIC, ELISA assay, and HPLC.

The immune-stimulatory conjugates as described herein can activate, stimulate or augment an immune response against a cell of a disease of condition. The activation, stimulation or augmentation of an immune response by an immune-stimulatory conjugate, such as a myeloid cell agonist, can be measured in vitro by co-culturing immune cells (e.g., myeloid cells) with cells targeted by the conjugate and measuring cytokine release, chemokine release, proliferation of immune cells, upregulation of immune cell activation markers, and/or ADCC. ADCC can be measured by determining the percentage of remaining target cells in the co-culture after administration of the conjugate with the target cells, myeloid cells, and other immune cells. In some embodiments, an immune-stimulatory conjugate can activate or stimulate immune cell activity, as determined by in vitro assay, such as a cytokine release assay, by detection of activation markers (e.g., WIC class II markers) or other assays known in the art. In some embodiments, an immune-stimulatory conjugate has an EC50 of 100 nM or less, as determine by cytokine release assay. In some embodiments, an immune-stimulatory conjugate has an EC50 of 50 nM or less, as determine by cytokine release assay. In some embodiments, an immune-stimulatory conjugate has an EC50 of 10 nM or less, as determine by cytokine release assay. In some embodiments, an immune-stimulatory conjugate has an EC50 of 1 mM or less.

### Immune-Stimulatory Compounds

The anti-mesothelin antibodies described herein can be conjugated via a linker to an immune-stimulatory compound in order to form an immune-stimulatory conjugate. An immune-stimulatory compound can be any compound that directly or indirectly stimulates an anti-tumor immune response after administration. For example, an immune-stimulatory compound can directly stimulate an anti-tumor immune response by causing the release of cytokines by its target cell, which results in the activation of immune cells. As another example, an immune-stimulatory compound can indirectly stimulate an immune response by suppressing IL-10 production and secretion by the target cell and/or by suppressing the activity of regulatory T cells, resulting in an increased anti-tumor response by immune cells. The stimulation of an immune response by an immune-stimulatory compound can be measured by the upregulation of proinflammatory cytokines and/or increased activation of immune cells. This effect can be measured in vitro by co-culturing immune cells with cells targeted by the immune-stimulatory conjugate and measuring cytokine release, chemokine release, proliferation of immune cells, upregulation of immune cell activation markers, and/or ADCC. ADCC can be measured by an ADCC assay, which can determine the percentage of remaining target cells, such as tumor cells, in the co-culture after administration of the immune-stimulatory conjugate with the target cells and immune cells.

In certain embodiments, an immune-stimulatory compound can target a pattern recognition receptor (PRR). PRRs can recognize pathogen-associated molecular patterns (PAMPs) and damage-associated molecular patterns (DAMPs). A PRR can be membrane bound. A PRR can be cytosolic. A PRR can be a toll-like receptor (TLR). A PRR can be RIG-I-like receptor. A PRR can be a receptor kinase. A PRR can be a C-type lectin receptor. A PRR can be a NOD-like receptor. A PRR can be TLR1, TLR2, TLR3, TLR4, TLRS, TLR6, TLR7, TLR8, TLR9, TLR10, TLR11, TLR12 or TLR13. A PRR can be TLR1, TLR2, TLR3, TLR4, TLRS, TLR6, TLR7, TLR8, TLR9, and TLR10.

In certain embodiments, the immune-stimulatory compound can be a Damage-Associated Pattern Molecule (DAMP) or a Pathogen-Associated Molecular Pattern Molecule (PAMP). Immune-stimulatory molecular motifs, such as PAMPs, can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as pathogens. Similar to other protein families, TLRs can have many isoforms, including TLR4, TLR7 and TLR8. TLR agonists can range from simple molecules to complex macromolecules. Likewise, the sizes of TLR agonists can range from small to large. TLR agonists can be synthetic or biosynthetic agonists. TLR agonists can also be PAMPs. Additional immune-stimulatory compounds, such as cytosolic DNA and unique bacterial nucleic acids called cyclic dinucleotides, can be recognized by Interferon Regulatory Factor (IRF) or stimulator of interferon genes (STING), which can act a cytosolic DNA sensor. Compounds recognized by Interferon Regulatory Factor (IRF) can play a role in immunoregulation by TLRs and other pattern recognition receptors.

The immune-stimulatory compound can comprise an inhibitor of TGFB, Beta-Catenin, PI3K-beta, STAT3, IL-10, IDO or TDO. The immune-stimulatory compound can be an inhibitor of the beta-catenin pathway, such as an inhibitor of TNIK or Tankyrase. In certain embodiments, the immune-stimulatory compound be a kinase inhibitor. In certain embodiments, the kinase inhibitor can be an inhibitor of CDK4/6, such as, for example, abemaciclib or palbociclib.

In some aspects, the immune-stimulatory compound is a myeloid cell agonist, for example, a TLR7 or TLR8 agonist. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine, but is other than a guanosine analog, an adenosine analog, a thymidine homopolymer, ssRNA, CpG-A, PolyG10, and PolyG3. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences). In some embodiments, a TLR7 agonist has an EC50 value of 500 nM or less by PBMC assay measuring TNFalpha or IFNalpha production. In some embodiments, a TLR7 agonist has an EC50 value of 100 nM or less by PBMC assay measuring TNFalpha or IFNalpha production. In some embodiments, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

In certain embodiments, the TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In certain embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine and is other a ssRNA. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463. In some embodiments, a TLR8 agonist has an EC50 value of 500 nM or less by PBMC assay measuring TNFalpha production. In some embodiments, a TLR8 agonist has an EC50 value of 100 nM or less by PBMC assay measuring TNFalpha production. In some embodiments, a TLR8 agonist has an EC50 value of 50 nM or less by PBMC assay measuring TNFalpha production. In some embodiments, a TLR8 agonist has an EC50 value of 10 nM or less by PBMC assay measuring TNFalpha production.

In some embodiments, a TLR8 agonist is any of the compounds described herein or in WO 2018/170179.

Other TLR7 and TLR8 agonists are disclosed in, for example, WO 2016142250, WO2017046112, WO2007024612, WO2011022508, WO2011022509, WO2012045090, WO2012097173, WO2012097177, WO2017079283, US20160008374, US20160194350, US20160289229, US Patent No. 6043238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics), WO2018198091(Novartis AG), and US20170131421 (Novartis AG).

In some aspects, the TLR8 agonist is a compound of Formula I:

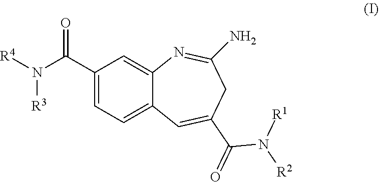

or a pharmaceutically acceptable salt thereof,
wherein:
R¹, R² and R³ are independently selected from hydrogen, optionally substituted C₁₋₁₀ alkyl, optionally substituted C₂₋₁₀ alkenyl, optionally substituted C₂₋₁₀ alkynyl, optionally substituted C₃-12 carbocycle, and optionally substituted 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and halo(C₁₋₁₀ alkyl);
R⁴ is an optionally substituted fused 5-5, fused 5-6, or fused 6-6 bicyclic heterocycle, and wherein optional substituents are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In some such aspects, le and R² are independently selected from Ci-4 alkyl and R³ is -hydrogen. In some such aspects, R¹ and R² are C₃ alkyl.

When attached to a linker, the linker is preferably bound to R⁴ or an optional substituent of R⁴.

In certain embodiments, an exemplary immune-stimulatory conjugate prior to attachment to the linker is represented by Formula (IV)

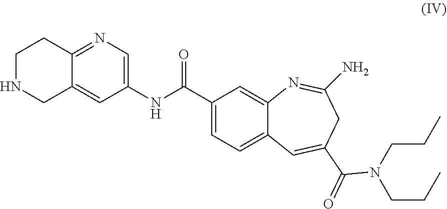

or a salt thereof. When attached to a linker, the exemplary immune-stimulatory can represented by Formula (IA)

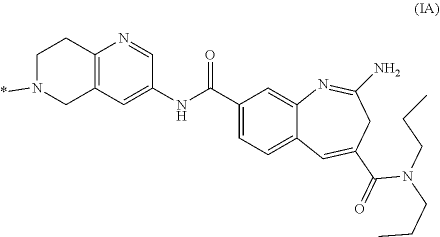 [SMILES: *N1CCc2ncc(NC(=O)c3ccc4c(c3)N=C(N)CC(C(=O)N(CCC)CCC)=C4)cc2C1]

wherein * indicates point of attachment to the linker.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Synthetic chemistry transformations and methodologies useful in synthesizing the compounds described herein are known in the art and include, for example, U.S. Pat. No. 10,239,862, incorporated herein by reference and for all purposes.

### Linkers

The anti-mesothelin antibodies described herein can be conjugated via a linker to a drug, e.g., an immune-stimulatory compound or cytotoxic agent.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker can, for example, comprise a peptide such as a valine-citrulline peptide or a valine-alanine peptide. A peptide-containing linker, such as a valine-citrulline- or valine-alanine-containing linker, can further contain, for example, a pentafluorophenyl group. A peptide-containing linker, such as a valine-citrulline- or valine-alanine-containing linker, can contain, for example, a maleimide or succinimide group. A peptide-containing linker, such as a valine-citrulline- or valine-alanine-containing linker, can further contain, for example, a para aminobenzyl alcohol (PABA) group or para-aminobenzyl carbamate (PABC). A peptide-containing linker, such as a valine-citrulline- or valine-alanine-containing linker, can contain, for example, a PABA group and a pentafluorophenyl group. A peptide-containing linker, such as a valine-citrulline- or valine-alanine-containing linker can contain, for example, a PABA group and a maleimide or succinimide group.

A non-cleavable linker can be protease insensitive. A non-cleavable linker can be a maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly) linker. A linker can be a linker suitable for attachment to an engineered cysteine or to a naturally occurring cysteine.

A linker can also comprise alkylene, alkenylene, alkynylene, polyether, polyester, polyamide group(s) and also, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker.

As will be appreciated by skilled artisans, the linkers may link a drug as described herein to an anti-mesothelin antibody by a covalent linkage between the linker and the antibody and compound.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can, in some aspects, rely on processes inside the cell to liberate a compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate drug release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the drug once the conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites.

Cleavable linkers can also include a disulfide group. Disulfides can be thermodynamically stable at physiological pH and can be designed to release the drug upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing drug in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a drug from a conjugate can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, cathepsin S, β-glucuronidase, or β-galactosidase.

The cleavable peptide can be selected, for example, from tetrapeptides or dipeptides such as Val-Cit, Val-Ala, and Phe-Lys. Dipeptides can have lower hydrophobicity compared to longer peptides.

Enzymatically cleavable linkers can include a self-immolative spacer to spatially separate the drug from the site of enzymatic cleavage. The direct attachment of a drug to a peptide linker can result in proteolytic release of the drug. The use of a self-immolative spacer can allow for the elimination of the fully active, chemically unmodified drug upon amide bond hydrolysis.

One self-immolative spacer can be a bifunctional para-aminobenzyl alcohol group, which can link to the peptide through the amino group, forming an amide bond, while amine containing drug can be attached through carbamate functionalities to the benzylic hydroxyl group of the linker (to give ap-amidobenzylcarbamate, PABC). The resulting pro-drug compound can be activated upon protease-mediated cleavage, leading to a 1,6-elimination reaction releasing the unmodified drug, carbon dioxide, and remnants of the linker. The following scheme depicts the fragmentation of p-amidobenzyl carbamate and release of the drug:

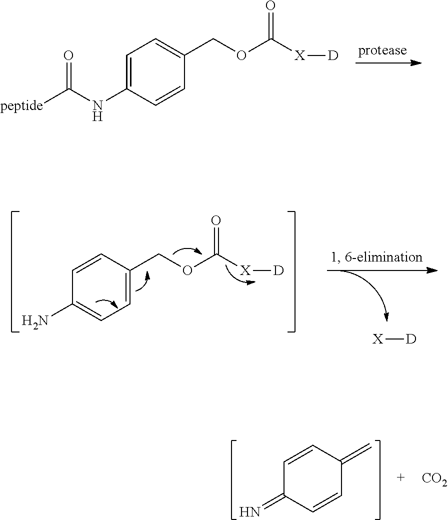

wherein X-D represents the unmodified drug.

The enzymatically cleavable linker can be a B-glucuronic acid-based linker. Facile release of the drug can be realized through cleavage of the B-glucuronide glycosidic bond by the lysosomal enzyme B-glucuronidase. This enzyme can be abundantly present within lysosomes and can be overexpressed in some tumor types, while the enzyme activity outside cells can be low. ß-Glucuronic acid-based linkers can be used to circumvent the tendency of an conjugate to undergo aggregation due to the hydrophilic nature of B-glucuronides.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a drug, wherein such ester groups can hydrolyze under physiological conditions to release the drug. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

An exemplary cleavable linker is represented by formula (V):

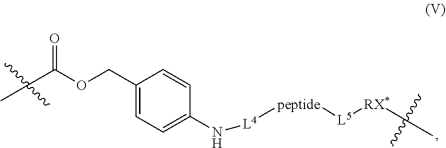

wherein L⁴ represents the C-terminal of the peptide and L⁵ is selected from a bond, alkylene and heteroalkylene, wherein L⁵ is optionally substituted with one or more groups independently selected from R³²; RX* comprises a bond, a succinimide moiety, or a hydrolyzed succinimide moiety bound to a residue of the antibody, wherein

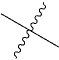

on RX* represents the point of attachment to the residue of

the antibody and the other

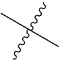

represents the point of attachment to the drug; and R³² is independently selected at each occurrence from halogen, —OH, —CN, —O—C₁₋₁₀ alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O—C₁₋₁₀ alkyl, —SH, ═O, ═S, —NH₂, —NO₂. In some such embodiments, the peptide of the linker is Val-Cit or Val-Ala.

In some such aspects, an exemplary cleavable linker is represented by formula (VI) or (VII)::

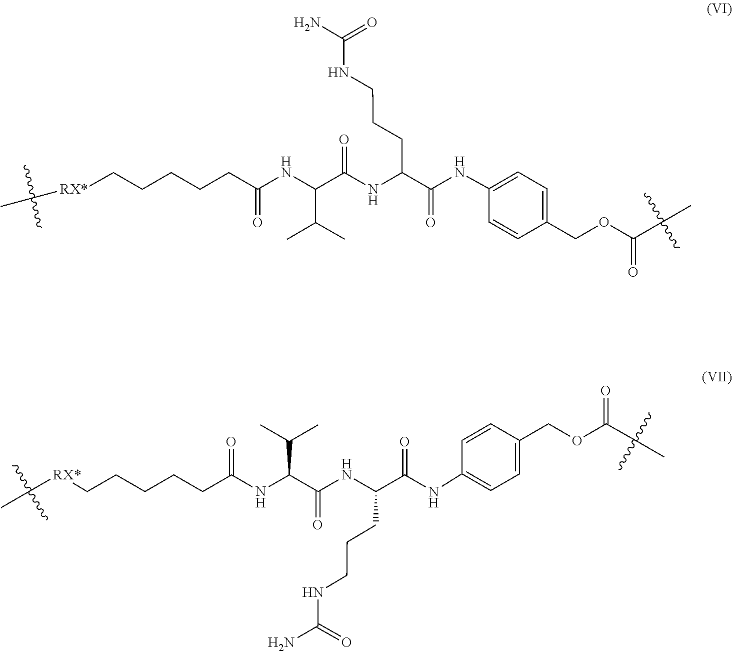

In some such aspects, an exemplary TLR8 agonist attached to linker is represented by Formulas (VIII) or (IX) or a pharmaceutically acceptable salt thereof:

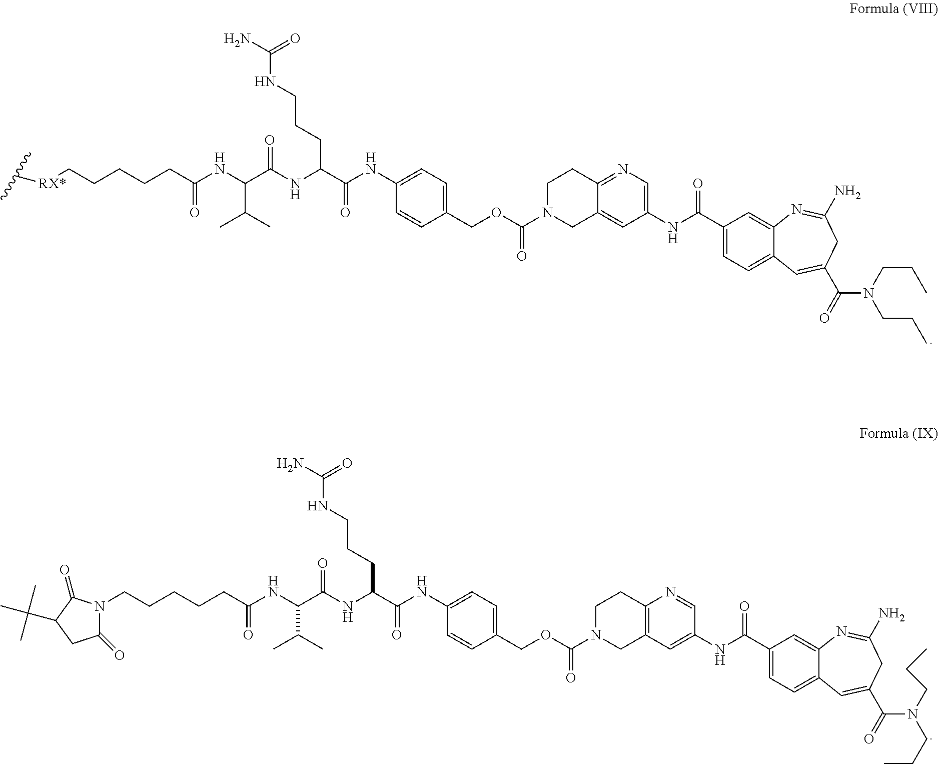

Although cleavable linkers can provide certain advantages, the linkers in the conjugates described herein need not be cleavable. For non-cleavable linkers, the drug release may not depend on the differential properties between the plasma and some cytoplasmic compartments. The release of the drug can occur, for example, after internalization of the conjugate via antigen-mediated endocytosis and delivery to lysosomal compartment, where the antibody can be degraded drug derivative, which is formed by the drug, the linker, and the amino acid residue or residues to which the linker was covalently attached. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

Attachment groups that are used to attach the linkers to an antibody can be electrophilic in nature and include, for example, maleimide groups, alkynes, alkynoates, allenes and allenoates, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reaction between a thiol group of an antibody and a drug with a linker including a maleimide group proceeds according to the following scheme:

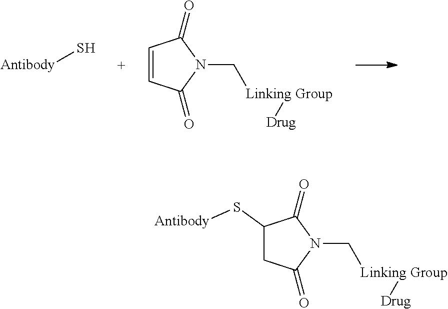

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. A representative schematic is shown below:

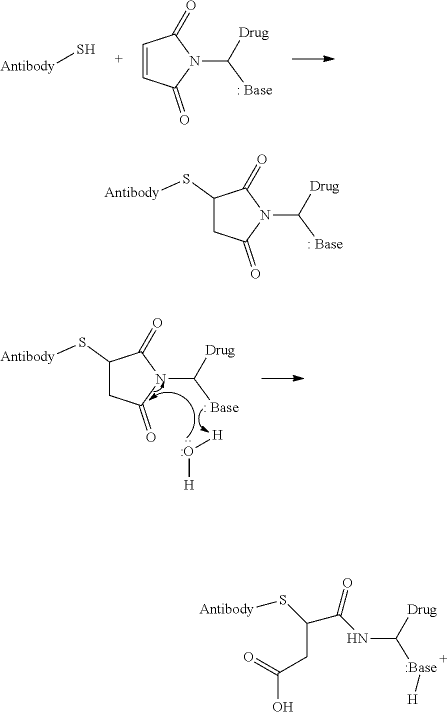

The hydrolysis reaction schematically represented above may occur at either carbonyl group of the succinimide group. Accordingly, two possible isomers may result, as shown below:

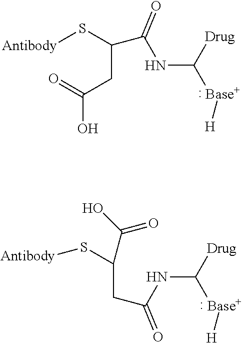

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate. Examples of self-stabilizing linkers are provided in, e.g., U.S. Patent Publication Number 2013/0309256, the linkers of which are incorporated by reference herein. It will be understood that a self-stabilizing linker useful in conjunction with the compounds of the present invention may be equivalently described as unsubstituted maleimide-including linkers, thio-substituted succinimide-including linkers, or hydrolyzed, ring-opened thio-substituted succinimide-including linkers.

### Attachment of Linkers to Antibodies

A linker may be bound to an antibody by a bond between the antibody and the linker. A linker may be bound to a terminus of an amino acid sequence of an antibody, or could be bound to a side chain modification to the antibody, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain of an antibody, or may be bound to a side chain modification of an Fc domain of an antibody, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain of an antibody, or may be bound to a side chain modification of an Fc domain of an antibody, such as the side chain of a lysine, serine, threonine, cysteine, tyrosine, aspartic acid, glutamine, a non-natural amino acid residue, or glutamic acid residue.

A linker may be bound to an antibody at a hinge cysteine. A linker may be bound to an antibody at a light chain constant domain lysine. A linker may be bound to an antibody at a heavy chain constant domain lysine. A linker may be bound to an antibody at an engineered cysteine in the light chain. A linker may be bound to an antibody at an Fc domain lysine. A linker may be bound to an antibody at an Fc domain cysteine. A linker may be bound to an antibody at a light chain glutamine, such as an engineered glutamine. A linker may be bound to an antibody at a heavy chain glutamine, such as an engineered glutamine. A linker may be bound to an antibody at an unnatural amino acid engineered into the light chain. A linker may be bound to an antibody at an unnatural amino acid engineered into the heavy chain. Amino acids can be engineered into an amino acid sequence of an antibody, for example, a linker of a conjugate. Engineered amino acids may be added to a sequence of existing amino acids. Engineered amino acids may be substituted for one or more existing amino acids of a sequence of amino acids.

A linker may be conjugated to an antibody via a sulfhydryl group on the antibody. A linker may be conjugated to an antibody via a primary amine on the antibody. A linker may be conjugated to an antibody via a residue of an unnatural amino acid on an antibody e.g., a ketone moiety.

### Lysine-based Bioconjugation

An antibody can be conjugated to a linker via lysine-based bioconjugation. An antibody can be exchanged into an appropriate buffer, for example, phosphate, borate, PBS, Tris-Acetate, Tris-Glycine, HEPES, MOPS, MES, EPS, HEPPS, Histidine, or HEPBS at a concentration of about 2 mg/mL to about 10 mg/mL. An appropriate number of equivalents of a drug-linker can be added as a solution with stirring. Dependent on the physical properties of the linker construct, a co-solvent can be introduced prior to the addition of the linker construct to facilitate solubility. The reaction can be stirred at room temperature for 2 hours to about 12 hours depending on the observed reactivity. The progression of the reaction can be monitored by LC-MS. Once the reaction is deemed complete, the remaining drug-linker constructs can be removed by applicable methods and the conjugate can be exchanged into the desired formulation buffer. Lysine-linked conjugates can be synthesized starting with antibody (mAb) or bispecific antibody (bsAb) and drug-linker construct, e.g., 10 equivalents, following Scheme A below. Monomer content and drug-antibody ratios (molar ratios) can be determined by methods described herein.

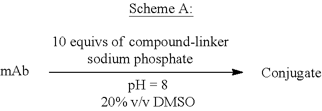

### Cysteine-based Bioconjugation

An antibody can be conjugated to a linker via cysteine-based bioconjugation. An antibody can be exchanged into an appropriate buffer, for example, phosphate, borate, PBS, Tris-Acetate, Tris-Glycine, HEPES, MOPS, MES, EPS, HEPPS, Histidine, or HEPBS at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A compound-linker described herein can be added as a solution with stirring. Dependent on the physical properties of the drug-linker construct, a co-solvent can be introduced prior to the addition of the drug-linker construct to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free drug-linker construct can be removed by applicable methods and the conjugate can be exchanged into the desired formulation buffer. Such cysteine-based conjugates can be synthesized starting with antibody (mAb) and drug-linker construct, e.g., 7 equivalents, using the conditions described in Scheme B below. Monomer content and drug-antibody ratios can be determined by methods described herein.

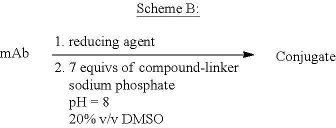

### Pharmaceutical Formulations

The conjugates described herein are useful as pharmaceutical compositions for administration to a subject in need thereof. Pharmaceutical compositions can comprise the conjugates described herein and one or more pharmaceutically acceptable excipients, suitable for administration to a subject. A pharmaceutical composition can further comprise buffers, carbohydrates, and/or preservatives, as appropriate. Pharmaceutical compositions comprising a conjugate can be manufactured, for example, by lyophilizing the conjugate, mixing, dissolving, emulsifying, encapsulating or entrapping the conjugate. The pharmaceutical compositions can also include the conjugates described herein in a free-base form or pharmaceutically-acceptable salt form.

Methods for formulation of the conjugates described herein can include formulating any of the conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the conjugates described herein can be lyophilized or in powder form for re-constitution with a suitable vehicle, e.g., sterile pyrogen-free water, before use.

The compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration, the conjugates can be formulated in a unit dosage injectable form (e.g., use letter solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the conjugate, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(-)-3-hydroxybutyric acid.

Pharmaceutical formulations described herein can be prepared for storage by mixing a conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Methods for formulation of the pharmaceutical compositions can include formulating any of the conjugates described herein with one or more inert, pharmaceutically-acceptable excipients or carriers to form a solid, semi-solid, or liquid composition for subcutaneous administration or for slow infusion IV administration. Solid compositions can include, for example, powders, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use. Formulations for subcutaneous administration have been described in, for example, WO2018/136412, WO2016/036678, WO2013/173687, WO2013/096835, WO2012/151199, WO2011/147921, WO2011/104381, WO2011/090088, WO2011/017070, WO2011/012637, WO2009/084659, and WO2004/091658, each of which is hereby incorporated by reference in its entirety. The conjugates can be formulated for subcutaneous administration in a unit dosage form in association with a pharmaceutically acceptable vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. The vehicle can contain minor amounts of additives such as substances that enhance isotonicity and chemical stability (e.g., buffers and preservatives).

The pharmaceutical compositions and formulations can be sterilized. Sterilization can be accomplished by filtration through sterile filtration.

Exemplary pharmaceutical compositions of the present invention can have an average drug load of, for example, from 1 to 20, 1 to 10, 1 to 8, 2 to 8, 2 to 5, 3 to 5 or 5 to 8.

### Therapeutic Applications

The conjugates and pharmaceutical compositions thereof are useful in the methods of the present disclosure for treating plurality of different subjects including, but not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The conjugates and pharmaceutical compositions thereof can be used in the methods described herein as a therapeutic, for example, as a treatment that can be administered in an effective regimen to a subject in need thereof to achieve a therapeutic effect. A therapeutic effect can be obtained in a subject by reduction, suppression, remission, alleviation or eradication of a disease state, including, but not limited to, one or more symptoms thereof. A therapeutic effect in a subject having a disease or condition, or exhibiting an early symptom thereof or exhibiting or otherwise suspected of being in or approaching an early stage of a disease or condition, can be obtained by a reduction, a suppression, a prevention, a delay, a remission, an alleviation or an eradication of the condition or disease, or pre-condition or pre-disease state.

It has been determined that when TLR agonists (e.g., TLR7 and TLR8 agonists) are administered as immune-stimulatory conjugates to a subject, the mode of delivery can be important. In certain instances, bolus repetitive IV administration can lead to anaphylaxis toxicities. The present inventors have discovered that if the immune-stimulatory conjugate is administered in a manner that results in a Tmax of greater than 4 hours following each dose, there is a reduced likelihood of anaphylaxis toxicity as compared to administration that results in a quicker Tmax. Generally, anaphylaxis-like toxicity associated with bolus repetitive IV administration is not observed until a subsequent dose is administered at least 7 or 8 days after administration of the first dose. That is, multiple doses may be administered for the first about 7 days without causing anaphylaxis-like toxicity, but a subsequent dose administered after about 7 days can cause anaphylaxis-like toxicity.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against a disease (mesothelin-expressing disease) treatable with a TLR agonist. The antibody of the conjugate recognizes an antigen associated with the disease or disease state.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against cell of a disease of condition. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against cancer cells, where the cancer cells express a mesothelin antigen.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against tumor cells of a solid tumor that expresses the mesothelin antigen.

One of ordinary skill in the art would understand that the amount, duration and frequency of administration of a pharmaceutical composition or conjugate described herein to a subject in need thereof depends on several factors including, for example but not limited to, the health of the subject, the specific disease or condition of the subject, the grade or level of a specific disease or condition of the subject, the additional therapeutics the subject is being or has been administered, and the like.

In some aspects of practicing the methods described herein, the immune-stimulatory conjugates are subcutaneously administered or administered by a slow IV infusion in an effective regimen of at least two or at least three cycles. Each cycle can optionally include a resting stage between cycles. Cycles of administration can be of any suitable length. In some embodiments, each cycle is a week (7 days), 10 days, every two weeks (14 days or biweekly), every three week (21 days) or every four weeks (28 days). In some embodiments, each cycle is a month. In some embodiments, at least two doses of the immune-stimulatory conjugate are administered more than 7 days apart, or more than 10 days apart. In some embodiments, at least one dose of the immune-stimulatory conjugate is administered more than 7 days, or more than 10 days, after the initial dose of the immune-stimulatory conjugate.

The dose of immune-stimulatory conjugate or pharmaceutical composition thereof within each cycle is an amount suitable to achieve a therapeutic effect. The dose within a cycle can be a single dose or a split dose (i.e., multiple doses within a cycle). In some embodiments, a split-dose is administered when the volume of the pharmaceutical composition to be administered is greater than is typically administered in a single dose by the selected route. For example, the maximum volume that is typically administered subcutaneously is about 1.5 mL, because greater volumes are believed to be associated with injection site pain and other adverse events at the injection site. Accordingly, in some embodiments, when the amount of the pharmaceutical composition to be administered subcutaneously is greater than about 1.5 mL, a split-dose is administered, meaning the volume is split into smaller volumes of, for example, less than 1.5 mL each, and the smaller volumes are each injected at a different site on the body of the subject. In certain embodiments, the total dose of immune-stimulatory conjugate or pharmaceutical composition thereof within a cycle is from about 0.1 to about 10 mg/kg. In some embodiments, the total dose is from about 0.5 to about 7.5 mg/kg. In some embodiments, the total dose is from about 0.5 to about 5 mg/kg. In some embodiments, the total dose is from about 0.5 to about 4 mg/kg. In some embodiments, the total dose is from about 0.5 to about 3.5 mg/kg. In some embodiments, the total dose is from about 0.5 to about 2 mg/kg.

The methods disclosed herein, using the immune stimulatory conjugates disclosed herein, include sequential administration (e.g., sequential subcutaneous administration) of a plurality of doses of immune stimulatory conjugates. This sequential administration avoids toxicities associated with repetitive bolus administration of the immune stimulatory conjugates. In some aspects, the immune stimulatory conjugates are administered in an effective regimen that results in a Tmax of the immune-stimulatory conjugate in the subject of greater than 4 hours following each administration of the immune-stimulatory conjugate. In some embodiments, the effective regimen results in a Tmax greater than 6 hours, greater than 8 hours, greater than 10 hours, greater than 12 hours, or greater than 15 hours following each administration of the immune-stimulatory conjugate. In some aspects, the Tmax is reached at or prior to 72 hours, at or prior to 48 hours, at or prior to 30 hours, at or prior to 24 hours, or at or prior to 16 hours.

Some treatment regimens can include, for example, a first subcutaneous or intravenous slow-infusion administration of an immune stimulatory conjugate, such as those disclosed herein, so as to elicit an initial targeted immune response as desired, against mesothelin-expressing cells. The treatment regimens then can include, for example, a second administration of an immune stimulatory conjugate through subcutaneous or intravenous slow-infusion administration. As disclosed herein, such as second administration comprises subcutaneous or intravenous slow-infusion administration of the immune stimulatory conjugate.

In some embodiments, B cells are deplated prior to administration of the immune-stimulatory conjugate. In some embodiments, an immune stimulatory conjugate is administered with a B-cell depleting agent. The B-cell depleting agent may be administered prior to, at the same time as, or after the immune stimulatory conjugate. The B-cell depleting agent may be administered, for example, within 14 days, within 7 days, within 1 day, within 24, 12, 6, 4, 3, 2, or 1 hour of the first administration of the immune-stimulatory conjugate. B-cell depleting agents include, but are not limited to, anti-CD20 antibodies, anti-CD19 antibodies, anti-CD22 antibodies, anti-BLyS antibodies, TACI-Ig, BR3-Fc, and anti-BR3 antibodies. Nonlimiting exemplary B-cell depleting agents include rituximab, ocrelizumab, ofatumumab, epratuzumab, MEDI-51 (anti-CD19 antibody), belimumab, BR3-Fc, AMG-623, and atacicept.

In some embodiments, the immune-stimulatory conjugate is administered with an agent that mitigates an anaphylactic-like toxicity. Nonlimiting exemplary agents that mitigate an anaphylactic-like toxicity include epinephrine, an antihistamine, a cortisone, and a beta-agonist. Administration may be, for example, within 1 hour or within minutes of administration of the immune-stimulatory conjugate.

Subcutaneous administration of immune stimulatory conjugate or slow IV infusion administration may be performed so as to spare or alleviate toxicities or avoid toxicities associated with repetitive bolus intravenous administration of the conjugate, such as an anaphylaxis-like response. A number of timing regimens are consistent with the second dose administration following first dose administration, such as administration of a second dose no more than 7, 8, 9, 10, 11, 12, 13, 14, 15, 16, 17, 18, 19, 20, 21, 22, 23, or 24 days after a first dose. Alternately, some dosage regimens comprise subcutaneous administration of a second dose at least 1, 2, 3, 4, 5, 6, 7, 8, 9, 10, 11, 12, 13, 14, 15, 16, 17, 18, 19, 20, 21, 22, 23, or 24 days after administration of a first dose.

Similarly, a number of dosage amounts are consistent with the methods disclosed herein. Typically, administration of a second dose and subsequent doses are at a level about or the same as that of a first dose. A second dose can variously greater than, equal to or less than a first dose. Dosage is often determined for a subject relative to an attribute of the subject, such as subject weight. Exemplary dosage amounts (e.g., subcutaneous dosage amounts) range, for example, from less than 1 mg/kg to 1 mg/kg, 2 mg/kg, 3 mg/kg, 4 mg/kg, 5 mg/kg, 6 mg/kg, 7 mg/kg, 8 mg/kg, 9 mg/kg, to 10 mg/kg and also contemplate values intermediate to those listed in the aforementioned range of values.

Methods disclosed herein may comprise monitoring a subject following administration of a first dose, a second dose, or one or more additional doses. A number of monitoring approaches are consistent with the disclosure herein. Monitoring is generally directed toward detection of at least one symptom or adverse event or at least one indicator of an increased risk of an anaphylaxis-like response. Exemplary monitoring comprises at least one monitoring process selected from a list comprising monitoring blood cell count, body temperature, skin discoloration, subject alertness or other indicator of anaphylaxis-like response.

Cancers and related disorders that can be treated or managed by methods and conjugates of the present invention include but are not limited to cancers of an epithelial cell origin. Examples of such cancers include the followings breast cancer including but not limited to ductal carcinoma, adenocarcinoma, lobular (small cell) carcinoma, intraductal carcinoma, medullary breast cancer, mucinous breast cancer. tubular breast cancer, papillary breast cancer, Paget's disease. triple-negative breast cancer, and inflammatory breast cancer; pancreatic cancer such as but not limited to, insulinoma, gastrinoma, glucagonoma, vipoma, somatostatin-secreting tumor, and carcinoid or islet cell tumor; vaginal cancers such as squamous cell carcinoma, adenocarcinoma. and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic. classic (typical), spermatocytic, nonseminoma, embryonal carcinoma, teratoma carcinoma, choriocarcinoma (yolk-sac tumor); oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. in addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia. and Murphy et al., 1997, Informed Decisions: The Complete Book of Cancer Diagnosis, Treatment, and Recovery, Viking Penguin, Penguin Books U.S.A., Inc., United States of America)

The methods and compositions of the invention are also useful in the treatment of a variety of cancers or other abnormal proliferative diseases, including (but not limited to) the following: carcinoma, including that of the bladder, breast, colon, kidney, liver, lung, ovary, pancreas, stomach, and cervix; including squamous cell carcinoma. it is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated in the skin, lung, colon, breast, prostate, bladder, kidney, pancreas, ovary, or uterus. In other specific embodiments, sarcoma, melanoma, or leukemia is treated.

In some embodiments, the cancer is malignant and overexpresses mesothelin. In other embodiments, the disorder to he treated is a pre-cancerous condition associated with cells that overexpress mesothelin.

Listing of Certain Sequences
Heavy chain Consensus Sequence
SEQ ID NO: 1
QVQLVQSGAE VKKPGSSVKV SCKASGX¹X²FX³ GYTMNWVRQA
PGQGLEWMGL ITPYNX⁴ASSY NQKFRGX⁵X⁶TX⁷ TX⁸DKSTSTAY
MELSSLRSED TAVYYCARGG YDGRGFDYWG QGTTVTVSS
X¹ is G or Y (G27Y)
X² is T or S (T28S)
X³ is S or T (S30T)
X⁴ is G or A (G55A)
X⁵ is R or K (R66K)
X⁶ is V or A (V67A)
X⁷ is I or L (I69L)
X⁸ is A or V (A71V)
VH1-e CDRgraft IgG1
SEQ ID NO: 2
QVQLVQSGAEVKKPGSSVKVSCKASGGTFSGYTMNWVRQAPGQGLEWMGL
ITPYNGASSYNQKFRGRVTITADKSTSTAYMELSSLRSEDTAVYYCARGG
YDGRGFDYWGQGTTVTVSS
VH1- G27Y, T28S, S30T
SEQ ID NO: 3
QVQLVQSGAE VKKPGSSVKV SCKASGYSFT GYTMNWVRQA
PGQGLEWMGL ITPYNGASSY NQKFRGRVTI TADKSTSTAY
MELSSLRSED TAVYYCARGG YDGRGFDYWG QGTTVTVSS
VH1-e R66K, V67A, I69L, A71V
SEQ ID NO: 4
QVQLVQSGAE VKKPGSSVKV SCKASGGTFS GYTMNWVRQA
PGQGLEWMGL ITPYNGASSY NQKFRGKATL TVDKSTSTAY
MELSSLRSED TAVYYCARGG YDGRGFDYWG QGTTVTVSS
VH1-e G27Y, T28S, S30T R66K, V67A, I69L,
A71V
SEQ ID NO: 5
QVQLVQSGAE VKKPGSSVKV SCKASGYSFT GYTMNWVRQA
PGQGLEWMGL ITPYNGASSY NQKFRGKATL TVDKSTSTAY
MELSSLRSED TAVYYCARGG YDGRGFDYWG QGTTVTVSS
VH1-e G27Y, T28S, S30T, R66K, V67A, A71V -
SEQ ID NO: 6
QVQLVQSGAE VKKPGSSVKV SCKASGYSFT GYTMNWVRQA
PGQGLEWMGL ITPYNGASSY NQKFRGKATI TVDKSTSTAY
MELSSLRSED TAVYYCARGG YDGRGFDYWG QGTTVTVSS
VH1-e G27Y, R66K, V67A, A71V -
SEQ ID NO: 7
QVQLVQSGAE VKKPGSSVKV SCKASGYTFS GYTMNWVRQA
PGQGLEWMGL ITPYNGASSY NQKFRGKATI TVDKSTSTAY
MELSSLRSED TAVYYCARGG YDGRGFDYWG QGTTVTVSS
VH1-e G27Y, G55A, R66K, V67A, A71V
SEQ ID NO: 8
QVQLVQSGAE VKKPGSSVKV SCKASGYTFS GYTMNWVRQA
PGQGLEWMGL ITPYNAASSY NQKFRGKATI TVDKSTSTAY
MELSSLRSED TAVYYCARGG YDGRGFDYWG QGTTVTVSS
VH1-e CDRgraft G55A
SEQ ID NO: 9
QVQLVQSGAE VKKPGSSVKV SCKASGGTFS GYTMNWVRQA
PGQGLEWMGL ITPYNAASSY NQKFRGRVTI TADKSTSTAY
MELSSLRSED TAVYYCARGG YDGRGFDYWG QGTTVTVSS
Light chain Consensus Sequence For
VKIII and for CDRs
SEQ ID NO: 10
X¹IVLTQSPATLSLSPGERATLSCSASSSVSYMEIWYQQKPGQAPRX²LI
YDTSKLASGIPARFSGSGSGTDFTLTISSLEPEDFAVYYCQQWSKHPLTF
GGGTKVEIK
X¹ is E or D (E1D)
X² is L or R (L46R)
VKIII-L6 CDRgraft
SEQ ID NO: 11
EIVLTQSPATLSLSPGERATLSCSASSSVSYMEIWYQQKPGQAPRLLIYD
TSKLASGIPARFSGSGSGTDFTLTISSLEPEDFAVYYCQQWSKHPLTFGG
GTKVEIK
VKIII-L6 E1D -
SEQ ID NO: 12
DIVLTQSPATLSLSPGERATLSCSASSSVSYMHWYQQKPGQAPRLLIYDT
SKLASGIPARFSGSGSGTDFTLTISSLEPEDFAVYYCQQWSKHPLTFGGG
TKVEIK
VKIII-L6 L46R
SEQ ID NO: 13
EIVLTQSPATLSLSPGERATLSCSASSSVSYMEIWYQQKPGQAPRRLIYD
TSKLASGIPARFSGSGSGTDFTLTISSLEPEDFAVYYCQQWSKHPLTFGG
GTKVEIK
VKIII-L6 E1D L46R
SEQ ID NO: 14
DIVLTQSPATLSLSPGERATLSCSASSSVSYMHWYQQKPGQAPRRLIYDT
SKLASGIPARFSGSGSGTDFTLTISSLEPEDFAVYYCQQWSKHPLTFGGG
TKVEIK
VKI-L12 CDRgraft
SEQ ID NO: 15
DIQMTQSPSTLSASVGDRVTITCSASSSVSYMHWYQQKPGKAPKWYDTSK
LASGVPSRFSGSGSGTEFTLTISSLQPDDFATYYCQQWSKHPLTFGGGTK
VEIK
CDR1 heavy chain
SEQ ID NO: 16
GYTMN
CDR2 heavy chain G55A
SEQ ID NO: 17
LITPYNAASSY NQKFRG
CDR2 heavy chain unmodified
SEQ ID NO: 18
LITPYNGASSY NQKFRG
CDR3 heavy chain
SEQ ID NO: 19
GGYDGRGFDY
CDR1 light chain
SEQ ID NO: 20
SASSSVSYMH
CDR2 light chain
SEQ ID NO: 21
DTSKLAS
CDR3 light chain
SEQ ID NO: 22
QQWSKHPLT
heavy chain constant region
SEQ ID NO: 23
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
light chain constant region
SEQ ID NO: 24
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
SS1 heavy chain variable region
SEQ ID NO: 25
QVQLQQSGPELEKPGASVKISCKASGYSFTGYTMNWVKQSHGKSLEWIGL
ITPYNGASSYNQKFRGKATLTVDKSSSTAYMDLLSLTSEDSAVYFCARGG
YDGRGFDYWGSGTPVTVSS
SS1 light chain variable region
SEQ ID NO: 26
DIELTQSPAIMSASPGEKVTMTCSASSSVSYMHWYQQKSGTSPKRWIYDT
SKLASGVPGRFSGSGSGNSYSLTISSVEAEDDATYYCQQWSKHPLTFGSG
TKVEIK
SS1 heavy chain variable region G55A
SEQ ID NO: 27
QVQLQQSGPELEKPGASVKISCKASGYSFTGYTMNWVKQSHGKSLEWIGL
ITPYNAASSYNQKFRGKATLTVDKSSSTAYMDLLSLTSEDSAVYFCARGG
YDGRGFDYWGSGTPVTVSS

### EXAMPLES

The following examples are included to further describe some embodiments of the present disclosure and should not be used to limit the scope of the disclosure.

### Example 1—Exemplary Humanized Antibodies

The human germline VH1-e with JH6 was used for CDR grafting the variable heavy chain and human germlines VKIII-L6 or VKI-L12 with JK4 were used for CDR grafting the variable light chain. CDR grafting was done using Kabat defined CDRs. Several variants containing mouse framework back mutations were generated, and the sequences determined using a 3D structural model for potential influence of residues on the CDR structure. Variable heavy region sequences were cloned into a vector containing a signal peptide sequence and IgG1 constant region. Variable light regions were cloned into a vector containing a signal peptide sequence and kappa constant region.

Six humanized heavy chains were co-transfected with 5 humanized light chains into the ExpiCHO expression system in a 30mL culture. The supernatant was analyzed via Octet to determine off rates and any that retained binding and contained less mouse sequence, were purified and further characterized. See Table 1. Clones 6 and 37 were selected for further optimization. An additional mutation in CDRH2, G55A, was introduced to reduce chemical modification of the neighboring Asparagine (N) residue. These humanized sequences are represented in SEQ ID NOs. 1-15.

TABLE 1

| Antibody | | | Titer | % | Sups | KD | Tm |
|---|---|---|---|---|---|---|---|
| Name | VH (IgG1) | VL (kappa) | mg/L | POI | Kdis | nM | ° C. |
| SS1 | VH | VL | 100-320 | 100 | 8.98E−09 | 945E−10- | 69 |
| | SEQ ID NO: 25 | SEQ ID NO: 26 | | | | 1.89E−09 | |
| 1 | VH1-e CDRgraft IgG1 | VKIII-L6 CDRgraft | 310 | 90 | 1.30E−08 | 2.29E−09 | 69 |
| | SEQ ID NO: 2 | SEQ ID NO: 11 | | | | | |
| 2 | VH1-e G27Y, T28S, S30T | VKIII-L6 t CDRgraft | 341 | 90 | 8.54E−09 | 1.75E−09 | 69 |
| | SEQ ID NOG | SEQ ID NO: 11 | | | | | |
| 3 | VH1-e R66K, V67A, I69L, A71V | VKIII-L6 CDRgraft | 148 | 94 | 6.55E−09 | 1.77E−09 | 70 |
| | SEQ ID NO: 4 | SEQ ID NO: 11 | | | | | |
| 4 | VH1-e G27Y, T28S, S30T R66K, | VKIII-L6 CDRgraft | 269 | 93 | 9.07E−09 | 1.44E−09 | 69 |
| | V67A, I69L, A71V | SEQ ID NO: 11 | | | | | |
| | SEQ ID NO: 5 | | | | | | |
| 5 | VH1-e G27Y, T28S, S30T, R66K, | VKIII-L6 CDRgraft | 214 | 92 | 8.59E−09 | 1.65E−09 | 70 |
| | V67A, A71V | SEQ ID NO: 11 | | | | | |
| | SEQ ID NO: 6 | | | | | | |
| 6 | VH1-e G27Y, R66K, V67A, A71V | VKIII-L6 CDRgraft | 291-420 | 95 | 9.65E−09 | 1.51E−09 | 70 |
| | SEQ ID NO: 7 | SEQ ID NO: 11 | | | | | |
| 7 | VH1-e CDRgraft IgG1 | VKIII-L6 E1D | 347 | | 1.02E−08 | | |
| | SEQ ID NO: 2 | SEQ ID NO: 12 | | | | | |
| 8 | VH1-e G27Y, T28S, S30T | VKIII-L6 E1D | 392 | | 8.94E−09 | | |
| | SEQ ID NO: 3 | SEQ ID NO: 12 | | | | | |
| 9 | VH1-e R66K, V67A, I69L, A71V | VKIII-L6 E1D | 263 | | 9.16E−09 | | |
| | SEQ ID NO: 4 | SEQ ID NO: 12 | | | | | |
| 10 | VH1-e G27Y, T28S, S30T R66K, | VKIII-L6 E1D | 305 | | 8.20E−09 | | |
| | V67A, I69L, A71V | SEQ ID NO: 12 | | | | | |
| | SEQ ID NO: 5 | | | | | | |
| 11 | VH1-e G27Y, T28S, S30T, R66K, | VKIII-L6 E1D | 301 | | 9.34E−09 | | |
| | V67A, A71V | SEQ ID NO: 12 | | | | | |
| | SEQ ID NO: 6 | | | | | | |
| 12 | VH1-e G27Y, R66K, V67A, A71V | VKIII-L6 E1D | 318 | | 7.27E−09 | | |
| | SEQ ID NO: 7 | SEQ ID NO: 12 | | | | | |
| 13 | VH1-e CDRgraft IgG1 | VKIII-L6 L46R | 253 | | 9.28E−09 | | |
| | SEQ ID NO: 2 | SEQ ID NO: 13 | | | | | |
| 14 | VH1-e G27Y, T28S, S30T | VKIII-L6 L46R | 331 | | 1.20E−08 | | |
| | SEQ ID NO: 3 | SEQ ID NO: 13 | | | | | |
| 15 | VH1-e R66K, V67A, I69L, A71V | VKIII-L6 L46R | 152 | | 7.97E−09 | | |
| | SEQ ID NO: 4 | SEQ ID NO: 13 | | | | | |
| 16 | VH1-e G27Y, T28S, S30T R66K, | VKIII-L6 L46R | 212 | | 1.04E−08 | | |
| | V67A, I69L, A71V | SEQ ID NO: 13 | | | | | |
| | SEQ ID NO: 5 | | | | | | |
| 17 | VH1-e G27Y, T28S, S30T, R66K, | VKIII-L6 L46R | 234 | | 8.90E−09 | | |
| | V67A, A71V | SEQ ID NO: 13 | | | | | |
| | SEQ ID NO: 6 | | | | | | |
| 18 | VH1-e G27Y, R66K, V67A, A71V | VKIII-L6 L46R | 244 | | 9.26E−09 | | |
| | SEQ ID NO: 7 | SEQ ID NO: 13 | | | | | |
| 19 | VH1-e CDRgraft IgG1 | VKIII-L6 E1D, L46R | 267 | | 1.15E−08 | 1.21E−09 | |
| | SEQ ID NO: 2 | SEQ ID NO: 14 | | | | | |
| 20 | VH1-e G27Y, T28S, S30T | VKIII-L6 E1D, L46R | 366 | | 1.12E−08 | 7.66E−10 | |
| | SEQ ID NO: 3 | SEQ ID NO: 14 | | | | | |
| 21 | VH1-e R66K, V67A, I69L, A71V | VKIII-L6 E1D, L46R | 206 | | 9.73E−09 | 8.67E−10 | |
| | SEQ ID NO: 4 | SEQ ID NO: 14 | | | | | |
| 22 | VH1-e G27Y, T28S, S30T R66K, | VKIII-L6 E1D, L46R | 419 | | 1.07E−08 | 6.23E−10 | |
| | V67A, I69L, A71V | SEQ ID NO: 14 | | | | | |
| | SEQ ID NO: 5 | | | | | | |
| 23 | VH1-e G27Y, T28S, S30T, R66K, | VKIII-L6 E1D, L46R | 310 | | 1.02E−08 | 8.84E−10 | |
| | V67A, A71V | SEQ ID NO: 14 | | | | | |
| | SEQ ID NO: 6 | | | | | | |
| 24 | VH1-e G27Y, R66K, V67A, A71V | VKIII-L6 E1D, L46R | 303 | | 1.15E−08 | 7.39E−10 | |
| | SEQ ID NO: 7 | SEQ ID NO: 14 | | | | | |
| 37 | VH1-e CDRgraft | VKI-L12 | 536 | 99 | | 1.21E−09 | 70 |
| | SEQ ID NO: 2 | SEQ ID NO: 15 | | | | | |
| 38 | VH1-e G27Y, T28S, S30T | VKI-L12 | 677 | 99 | | 7.66E−10 | 70 |
| | SEQ ID NO: 3 | SEQ ID NO: 15 | | | | | |
| 39 | VH1-e R66K, V67A, I69L, A71V | VKI-L12 | 642 | 99 | | 8.67E−10 | 71 |
| | SEQ ID NO: 4 | SEQ ID NO: 15 | | | | | |
| 40 | VH1-e G27Y, T28S, S30T R66K, | VKI-L12 | 514 | 100 | | 6.23E−10 | 70 |
| | V67A, I69L, A71V | SEQ ID NO: 15 | | | | | |
| | SEQ ID NO: 5 | | | | | | |
| 41 | VH1-e G27Y, T28S, S30T, R66K, | VKI-L12 | 578 | 100 | | 8.84E−10 | 70 |
| | V67A, A71V | SEQ ID NO: 15 | | | | | |
| | SEQ ID NO: 6 | | | | | | |
| 42 | VH1-e G27Y, R66K, V67A, A71V | VKI-L12 | 594 | 100 | | 7.39E−10 | 70 |
| | SEQ ID NO: 7 | SEQ ID NO: 15 | | | | | |
| 55 | VH1-e G27Y, G55A, R66K, | VKIII-L6 | 364 | 97 | | 4.60E−10 | 70 |
| | V67A, A71V | SEQ ID NO: 11 | | | | | |
| | SEQ ID NO: 8 | | | | | | |
| 56 | VH1-e CDRgraft G55A | VKI-L12 | 506 | 100 | | 1.32E−09 | 70 |
| | SEQ ID NO: 9 | SEQ ID NODS | | | | | |

### Example 2—Characterization of SS1 Humanized Clones by Hydrophobic Interaction Chromatography (HIC) and Liquid Chromatography-Mass Spectrometry (LC-MS)

Antibody SS1 consists of multiple variant species, as evident from its profile on an analytical HIC column (TSKgel Butyl-NPR with ammonium sulfate as the kosmotropic salt), while the four humanized binding domains (6, 37, 55 and 56 in Table 1) result in more homogeneous profiles. Among the 4 humanized binding domains, 55 and 56 undergo significantly fewer changes upon exposure to high temperature of 40° C. for up to 4 weeks as compared to 6 and 37, respectively, and differ from their parental humanized sequence only by a single mutation, G55A, engineered to reduce the potential deamidation of N54. The peptide mapping by LC-MS (using Charged Surface Hybrid C18 column) of samples subjected to thermal stress and digested by trypsin directly support that the 55 and 56 antibodies are significantly less prone to deamidation compared to the parental antibodies and therefore display increased stability.

### Example 3—Humanized Anti-mesothelin Antibodies Bound to Human MSLN Expressed on an Ovcar3 Tumor Cell Line

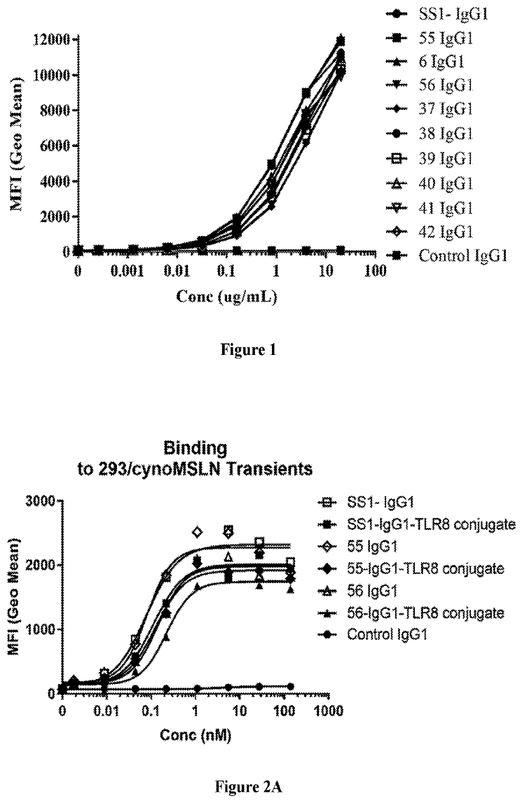

OVCAR3 cells (50,000/well) were incubated with titrating concentrations of unconjugated anti-MSLN antibodies or a control antibody (anti-digoxin) in FACS Wash (FW-PBS, 2.0% FBS, 1 mM EDTA) for 30 mins at 4° C. Cells were washed with FW, anti-huIgG1-PE added and incubated for a further 30 mins at 4° C. Cells were washed and analyzed by flow cytometry. FIG. 1 demonstrates that the humanized anti-mesothelin antibodies bound to human MSLN expressed on an Ovcar3 tumor cell line.

### Example 4—Exemplary Anti-mesothelin TLR8 Agonist Conjugates

Antibodies SS1, 6, 37, 55 and 56 were conjugated to drug-linker to form immunconjugates SS1-TLR8, 6-TLR8, 37-TLR8, 55-TLR8 and 56-TLR8, respectively. Each antibody was exchanged into HEPES buffer (100 mM, pH 7.0, 1 mM DTPA) at a concentration of 10 mg/mL. To each antibody solution was added 2.2 equivalents of the reducing agent tris(2-carboxyethyl)phosphine. The resultant solution was mixed gently at ambient temperature for 90 min. Upon completion of the reduction, to the antibody solution was added DMSO to a final concentration of 10% v/v. Next, drug-linker:

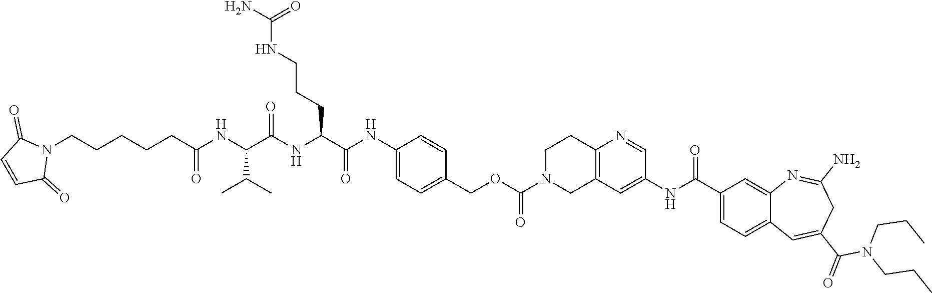 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

was added dropwise as a solution (7.0 equiv, 10 mM in DMSO). The resultant mixture was mixed gently at ambient temperature for 30 minutes, at which time the unreacted drug-linker was quenched via the addition of cysteine. The conjugate was then purified via preparative size exclusion chromatography and the conjugate was exchanged into the desired formulation buffer. Monomer content and drug-antibody ratios can be determined herein.

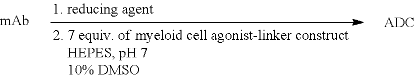

The TLR8 drug-linker was synthesized as described in U.S. Pat. No. 10,239,862.

### Example 5—Anti-mesothelin Immunoconjugates Bound to Human MSLN Expressing Tumor Cell Lines

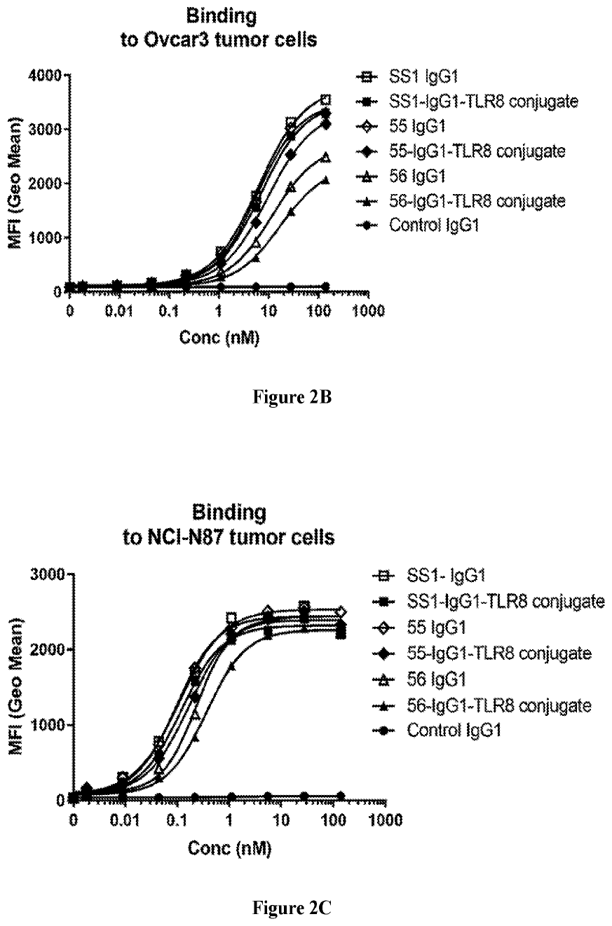

MSLN-expressing cell lines (293 cells transfected with cyno MSLN, OVCAR3 cells, and NCI-N87 cells at 25,000/well) were incubated with titrating concentrations of unconjugated anti-mesothelin antibodies (antibodies 55 or 56), a control antibody (anti-digoxin), or anti-mesothelin immunoconjugates in FACS Wash (FW-PBS, 2.05% FBS, 1mM EDTA) for 30 mins at 4° C. Cells were washed with FW, anti-huIgG1-PE added and incubated for a further 30 mins at 4° C. Cells were washed and analyzed by flow cytometry. FIGS. 2A-2C shows that the anti-mesothelin immunoconjugates bind to MSLN-expressing cells with a similar EC₅₀ as unconjugated anti-mesothelin antibodies.

### Example 6—TNFα Production by huPBMCs was Induced by Anti-mesothelin TLR8 Agonist Conjugates in the Presence of Cells Transfected with Human MSLN

### Materials and General Procedures

Human Whole Blood was obtained from Bloodworks Northwest and collected in 10mL EDTA tubes. Human PBMCs were then isolated from the whole blood by Ficoll gradient centrifugation and resuspended in assay media (RPMI-1640 Medium supplemented with 10% Fetal Bovine Serum, 1 mM Sodium Pyruvate, 1× GlutaMAX-1, 1× Non-Essential Amino Acids, 10mM HEPES and 0.5% Penicillin/Streptomycin; all from Gibco). Tumor cells were removed from tissue culture flasks with HyQTASE (Hyclone), washed twice and resuspended in assay media.

Human PBMCs isolated as described above were resuspended in assay media, and plated in 96-well flat bottom microtiter plates (125,000/well). MSLN-expressing HEK-293cells were then added (25,000/well) along with titrating concentrations of conjugated or unconjugated antibodies. Mock transfected HEK-293 cells were used as a negative control. After overnight culture, supernatants were harvested, and TNFα levels were determined by AlphaLISA.

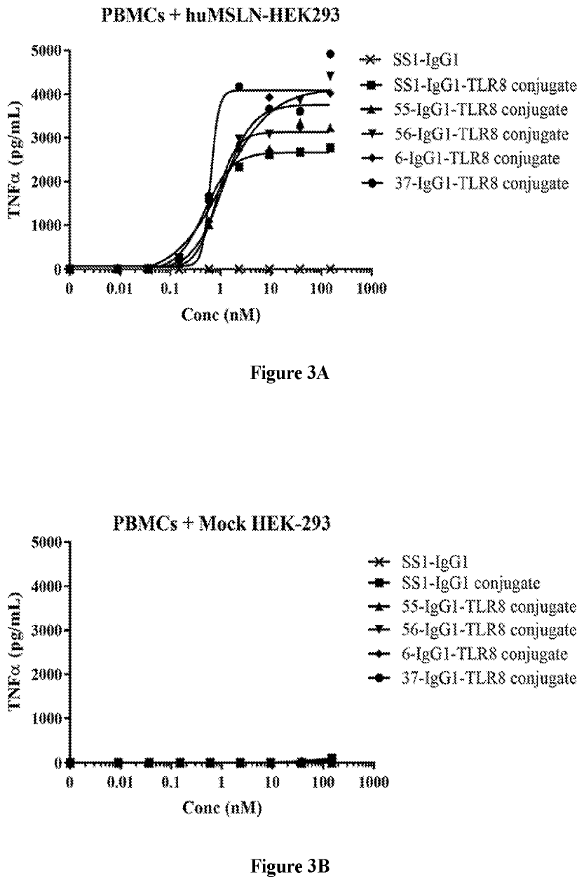

Referring to FIG. 3, all the immunoconjugates were active with HEK-293 cells transfected with human MSLN, stimulating production of TNFα from huPBMCs in a dose-dependent manner (FIG. 3A). In contrast, unconjugated anti-mesothelin antibody SS1 did not stimulate TNFα production from huPBMCs in the presence of MSLN-HEK-293 cells. Furthermore, none of the immunoconjugates or unconjugated antibody stimulated TNF-α production from huPBMCs in the presence of HEK-293 cells lacking expression of MSLN (FIG. 3B).

### Example 7—TNFα Production by huPBMCs was Induced by Anti-mesothelin TLR8 Agonist Conjugates in the Presence of Mesothelin Expressing Tumor Cell Lines

PBMCs were isolated from human blood as described above. Briefly, huPBMCs were isolated by Ficoll gradient centrifugation, resuspended in RPMI, and plated in 96-well flat bottom microtiter plates (125,000/well). MSLN-expressing tumor cells were then added (25,000/well) along with titrating concentrations of conjugated or unconjugated antibodies. Mock-transfected HEK-293 cells were used as a negative control. After overnight culture, supernatants were harvested, and TNFα levels were determined by AlphaLISA.

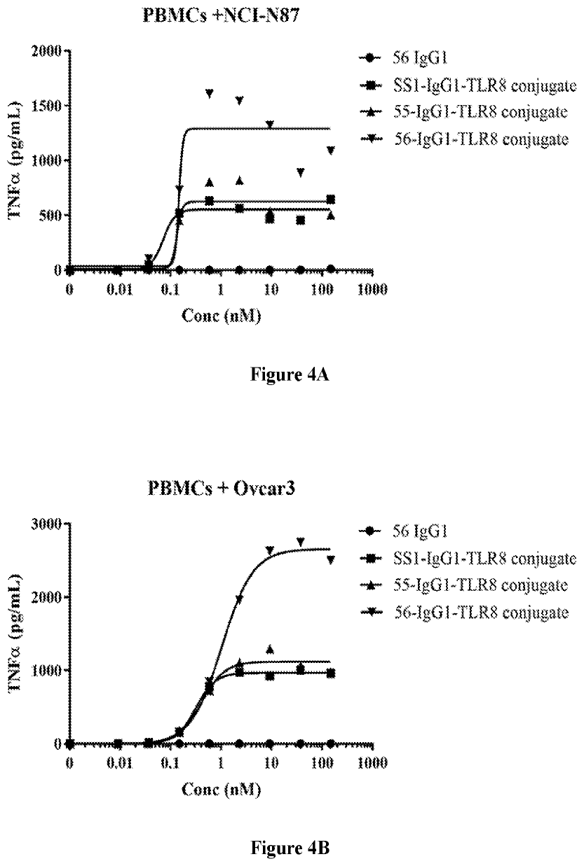

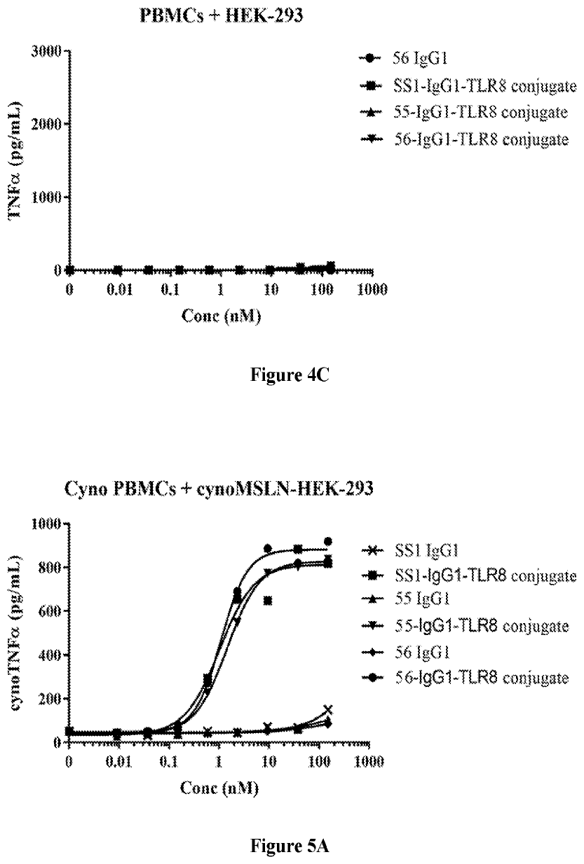

Referring to FIG. 4, anti-mesothelin TLR8 agonist conjugates induced TNFα production in a dose-dependent manner from huPBMCs in the presence of MSLN expressing tumor cells, NCI-N87 and OVCAR3 but not in the presence of the HEK-293 cells lacking expression of MSLN (FIG. 4A-C). Unconjugated anti-mesothelin antibody did not stimulate TNFα production from PBMCs in the presence of any of the tumor cell lines. Furthermore, none of the conjugated or unconjugated antibodies stimulated TNF-αproduction from PBMCs in the absence of MSLN-expressing tumor cells (Data not shown).

### Example 8—TNFα Production by Cyno PBMCs was Induced by Anti-mesothelin TLR8 Agonist Conjugates in the Presence of Tumor Cells Transfected with Human MSLN

Frozen cynomolgus monkey PBMCs were obtained from Primate Biological and stored in liquid nitrogen. For culture, cyno PBMC were thawed quickly in a 37° C. water bath and diluted into pre-warmed RPMI 1640 (Lonza) supplemented with 10% fetal bovine serum, 2 mM glutamine, 50 μg/mL penicillin, 50 U/mL streptomycin (all from Gibco) and centrifuged for 5 minutes at 500 x g. Cyno PBMCs were then resuspended in assay media for use.

PBMCs were plated in 96-well flat bottom microtiter plates (125,000/well). MSLN-expressing HEK-293 cells were then added (25,000/well) along with titrating concentrations of conjugated or unconjugated antibodies. Mock transfected HEK-293 cells were used as a negative control. After overnight culture, supernatants were harvested, and TNFα levels were determined by AlphaLISA.

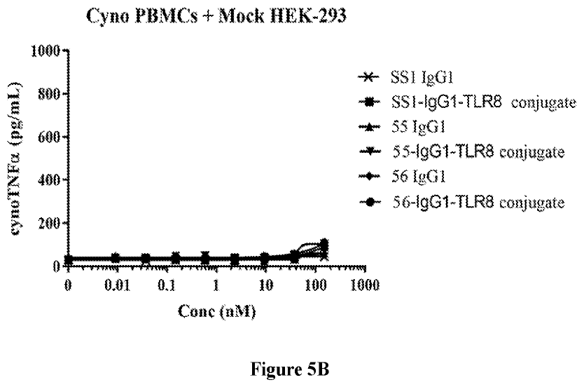

The anti-mesothelin TLR8 agonist conjugates were active with HEK-293 transfected with cyno MSLN, stimulating production of TNFα from cyno PBMCs in a dose-dependent manner. In contrast, unconjugated anti-mesothelin antibodies did not stimulate TNFα production from cyno PBMCs in the presence of MSLN-HEK-293 cells (FIG. 5A). Furthermore, none of the conjugated or unconjugated antibody stimulated TNFα production from cyno PBMCs in the presence of HEK-293 cells lacking expression of MSLN (FIG. 5B).

### Example 9—Administration of an Anti-mesothelin TLR8 Agonist Conjugate to Non-human Primates Results in Immune Activation

SS1-TLR8 was evaluated in a repeat-dose non-human primate (NHP) study to evaluate safety and pharmacodynamic (PD) effects. Animals were administered test article subcutaneously at 6 mg/kg Q3W for 3 doses. The conjugate was well tolerated, with no adverse clinical signs or reduction in body weights noted (data not shown). Clinical pathology changes present resolved to baseline, or were trending towards baseline, at the conclusion of the study and were not adverse. There were no significant findings at necropsy or during histopathological evaluations, including at tissues known to have mesothelin expression. After each dose, transient PD-related effects consistent with TLR8 agonist activity were noted in key parameters from clinical chemistry, hematology, and cytokine/chemokine production, including increases in CRP, MCP-1, MIP-1beta, neutrophils, and decreases in albumin and lymphocytes. These changes were indicative of mild to moderate activation of the innate immune system, without the clinical signs or inflammatory cytokine production associated with CRS (data not shown).

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed:

1. A conjugate comprising an antibody that specifically binds to human mesothelin conjugated via a linker to a compound of Formula (I): 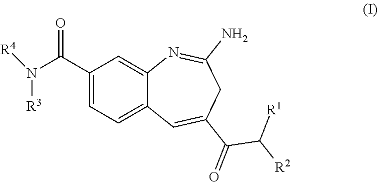
or a pharmaceutically acceptable salt thereof,
wherein:
R¹, R² and R³ are independently selected from hydrogen, optionally substituted C₁₋₁₀ alkyl, optionally substituted C₂₋₁₀ alkenyl, optionally substituted C₂₋₁₀ alkynyl, optionally substituted C₃₋₁₂ carbocycle, and optionally substituted 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and halo(C₁₋₁₀ alkyl);
R⁴ is an optionally substituted fused 5-5, fused 5-6, or fused 6-6 bicyclic heterocycle, and wherein optional substituents of R⁴ are independently selected at each occurrence from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.
wherein the linker is bound R⁴ or an optional substituent of R⁴; and wherein the antibody comprises a heavy chain variable region comprising complementarity determining regions (CDRs) having the amino acid sequences of the heavy chain variable region CDRs set forth in SEQ ID NO:1 and a light chain variable region comprising CDRs having the amino acid sequences of the light chain variable region CDRs set forth in SEQ ID NO:10.

2. The conjugate of claim 1 wherein the conjugate is represented by Formula (II): 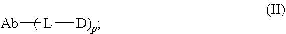
wherein:
Ab is the antibody,
L is the linker;
D is the compound of Formula (I) or a pharmaceutically acceptable salt thereof; and
p is from 1 to 20.

3. The conjugate of claim 1 or claim 2 wherein the compound of Formula (I) has Formula (IA): 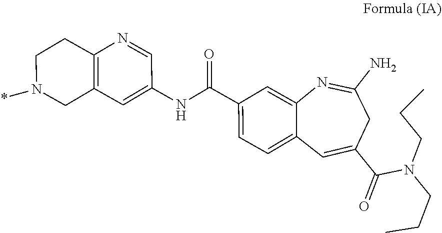 [SMILES: *N1CCc2ncc(NC(=O)c3ccc4c(c3)N=C(N)CC(C(=O)N(CCC)CCC)=C4)cc2C1]
or a pharmaceutically acceptable salt thereof, wherein * indicates point of attachment to the linker.

4. The conjugate of any one of claims 1-3, wherein the linker is a cleavable linker.

5. The conjugate of claim 4, wherein the linker is cleavable by a lysosomal enzyme.

6. The conjugate of any one of claims 1-5, wherein the linker is represented by formula (V): 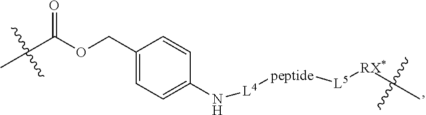
wherein L⁴ represents the C-terminal of the peptide and L⁵ is selected from a bond, alkylene and heteroalkylene, wherein L⁵ is optionally substituted with one or more groups independently selected from R³²; RX* comprises a bond, a succinimide moiety, or a hydrolyzed succinimide moiety bound to a residue of the antibody, wherein 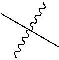
on RX* represents the point of attachment to the residue of the antibody and the other 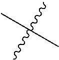
represents the point of attachment to the compound of Formula (I); and R³² is independently selected at each occurrence from halogen, —OH, —CN, —O—C₁ alkyl, —SH, ═O, ═S, —NH₂, —NO₂;
and C₁₋₁₀ alkyl, C₂₋₁₀ alkenyl, C₂₋₁₀ alkynyl, each of which is optionally substituted with one or more substituents independently selected from halogen, —OH, —CN, —O—C₁₋₁₀ alkyl, —SH, ═O, ═S, —NH₂, —NO₂.

7. The conjugate of claim 6, wherein the peptide of the linker is Val-Cit or Val-Ala.

8. The conjugate of claim 7, wherein the linker is represented by Formula (VI) or (VII):

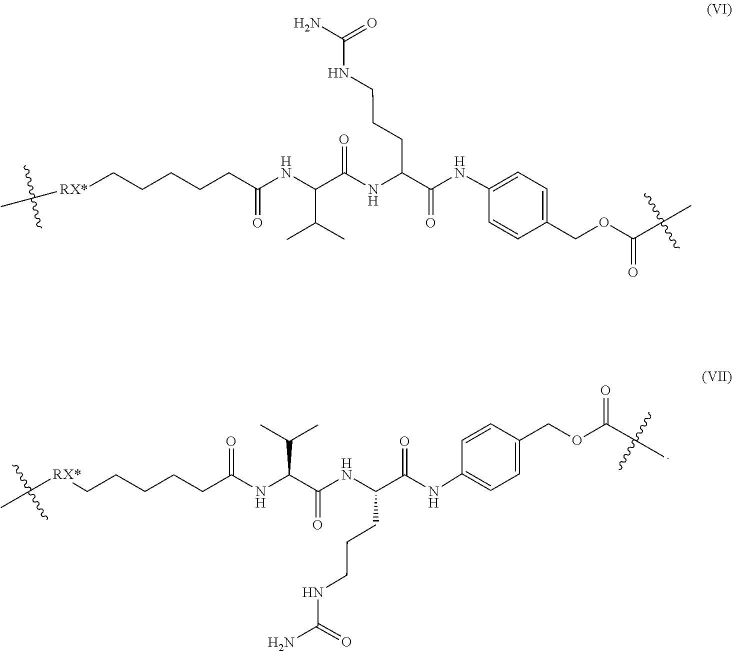

9. The conjugate of claim 3, wherein the linker and compound of Formula I is represented by Formula (VIII): 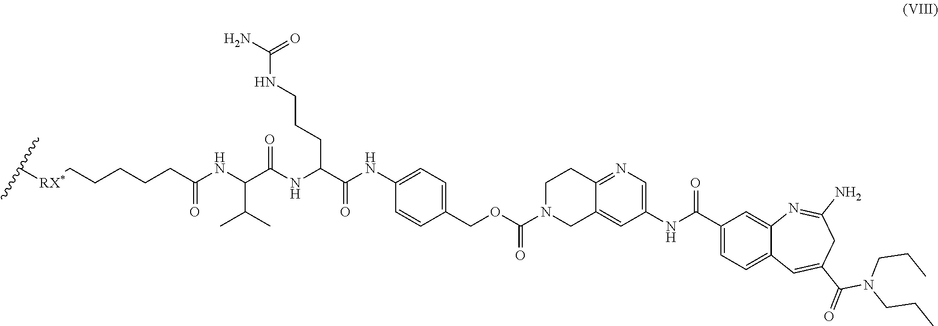
or a pharmaceutically acceptable salt thereof, wherein RX* comprises a bond, a succinimide moiety, or a hydrolyzed succinimide moiety bound to a residue of the antibody, wherein 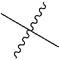
on RX* represents the point of attachment to the residue of the antibody.

10. The conjugate of claim 9, wherein the linker and compound of Formula I is represented by Formula (IX):

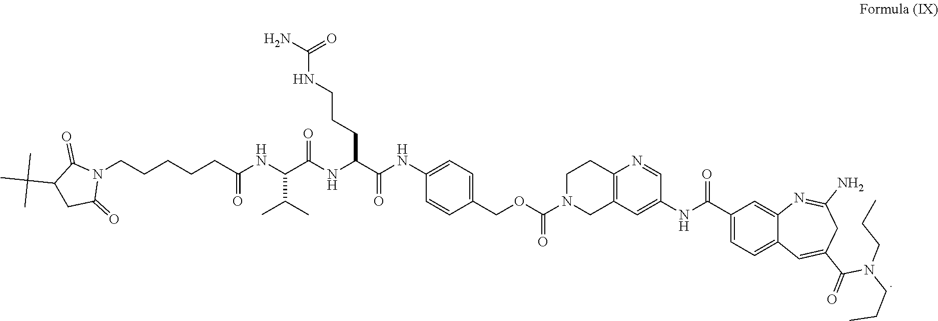 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)CC(C(C)(C)C)C5=O)C(C)C)cc3)CC4)cc2N=C(N)C1]

11. The conjugate of any one of claims 2 to 10, wherein p is from 1 to 8.

12. The conjugate of any one of claims 1 to 11, wherein the antibody is a humanized antibody.

13. The conjugate of any one of claims 1 to 12, wherein the antibody comprises a heavy chain variable region comprising complementarity determining regions (CDRs) having the amino acid sequences of the heavy chain variable region CDRs set forth in SEQ ID NO:9.

14. The conjugate of any one of claims 1 to 12, wherein the antibody comprises a heavy chain variable region comprising complementarity determining regions (CDRs) having the amino acid sequences of the heavy chain variable region CDRs set forth in SEQ ID NO:2.

15. The conjugate of any one of claims 1 to 14, wherein the CDR residues are identified according to Kabat.

16. The conjugate of any one of claims 1 to 12, wherein the antibody comprises a heavy chain (HC) CDR1 comprising the amino acid sequence of SEQ ID NO: 16, a HC CDR2 comprising the amino acid sequence of SEQ ID NO: 17 or 18, a HC CDR3 comprising the amino acid sequence of SEQ ID NO: 19, a light chain (LC) CDR1 comprising the amino acid sequence of SEQ ID NO: 20, a LC CDR2 comprising the amino acid sequence of SEQ ID NO: 21, and a LC CDR3 comprising the amino acid sequence of SEQ ID NO: 22.

17. The conjugate of any one of claims 1 to 16, wherein the antibody comprises a heavy chain variable region comprising the amino acid sequence set forth in SEQ ID NO:1.

18. The conjugate of claim 17, wherein the antibody comprises a heavy chain variable region comprising the amino acid sequence set forth in SEQ ID NO:2.

19. The conjugate of claim 17, wherein the antibody comprises a heavy chain variable region comprising the amino acid sequence set forth in SEQ ID NO:3.

20. The conjugate of claim 17, wherein the antibody comprises a heavy chain variable region comprising the amino acid sequence set forth in SEQ ID NO:4.

21. The conjugate of claim 17, wherein the antibody comprises a heavy chain variable region comprising the amino acid sequence set forth in SEQ ID NO:5.

22. The conjugate of claim 17, wherein the antibody comprises a heavy chain variable region comprising the amino acid sequence set forth in SEQ ID NO:6.

23. The conjugate of claim 17, wherein the antibody comprises a heavy chain variable region comprising the amino acid sequence set forth in SEQ ID NO:7.

24. The conjugate of claim 17, wherein the antibody comprises a heavy chain variable region comprising the amino acid sequence set forth in SEQ ID NO:8.

25. The conjugate of claim 17, wherein the antibody comprises a heavy chain variable region comprising the amino acid sequence set forth in SEQ ID NO:9.

26. The conjugate of any one of claims 1 to 25, wherein the antibody comprises a light chain variable region comprising the amino acid sequence set forth in SEQ ID NO:10.

27. The conjugate of claim 26, wherein the antibody comprises a light chain variable region comprising the amino acid sequence set forth in SEQ ID NO:11.

28. The conjugate of claim 26, wherein the antibody comprises a light chain variable region comprising the amino acid sequence set forth in SEQ ID NO:12.

29. The conjugate of claim 26, wherein the antibody comprises a light chain variable region comprising the amino acid sequence set forth in SEQ ID NO:13.

30. The conjugate of claim 26, wherein the antibody comprises a light chain variable region comprising the amino acid sequence set forth in SEQ ID NO:14.

31. The conjugate of claims 1 to 25 wherein the antibody comprises a light chain variable region comprising the amino acid sequence set forth in SEQ ID NO:15.

32. The conjugate of any one of claims 1 to 16, wherein the antibody comprises a heavy chain variable region comprising the amino acid sequence set forth in SEQ ID NO:1 and a light chain variable region comprising the amino acid sequence set forth in SEQ ID NO:10 or SEQ ID NO:15.

33. The conjugate of any one of claims 1 to 16, wherein the antibody comprises a heavy chain variable region comprising the amino acid sequence set forth in SEQ ID NO:8 and a light chain variable region comprising the amino acid sequence set forth in SEQ ID NO:11.

34. The conjugate of any one of claims 1 to 16, wherein the antibody comprises a heavy chain variable region comprising the amino acid sequence set forth in SEQ ID NO:9 and a light chain variable region comprising the amino acid sequence set forth in SEQ ID NO:11.

35. The conjugate of any one of claims 1 to 16, wherein the antibody comprises a heavy chain variable region comprising the amino acid sequence set forth in SEQ ID NO:9 and a light chain variable region comprising the amino acid sequence set forth in SEQ ID NO:15.

36. The conjugate of any one of claims 1 to 11, wherein the antibody comprises a heavy chain variable region comprising the amino acid sequence set forth in SEQ ID NO:25 or SEQ ID NO:27 and a light chain variable region comprising the amino acid sequence set forth in SEQ ID NO:26.

37. The conjugate of any one of claims 1 to 36, wherein the antibody is an IgG1 antibody.

38. The conjugate of any one of claims 1 to 37, wherein the antibody comprises a heavy chain constant region comprising the amino acid sequence set forth in SEQ ID NO:23 and a light chain constant region comprising the amino acid sequence set forth in SEQ ID NO:24.

39. The conjugate of any one of claims 1 to 36, wherein the antibody comprises a wild-type IgG1 Fc domain or an IgG1 Fc domain variant having the same or substantially similar binding affinity to FcγRI, FcγRII, and FcγRIII as compared to a wild-type IgG1 Fc domain.

40. The conjugate of any one of claims 1 to 36, wherein the antibody comprises a wild-type IgG1 Fc domain or an IgG1 Fc domain variant having the same or substantially similar binding affinity to FcRn as compared to a wild-type IgG1 Fc domain.

41. The conjugate of any one of claims 1 to 36, wherein the antibody comprises a wild-type IgG1 Fc domain or an IgG1 Fc domain variant having increased or decreased affinity to one or more Fcγ receptors as compared to a wild-type IgG1 Fc domain.

42. The conjugate of any one of claims 1 to 41, wherein the antibody is a full-length antibody.

43. The conjugate of any one of claims 1 to 41, wherein the antibody is an antigen binding fragment.

44. A pharmaceutical composition comprising a conjugate of any one of claims 1 to 43, and a pharmaceutically acceptable carrier.

45. The pharmaceutical composition of claim 44, wherein the average drug load of the composition is from 2 to 8.

46. The pharmaceutical composition of claim 44, wherein the average drug load of the composition is from 2 to 5.

47. A method of treating a mesothelin-expressing cancer, comprising administering to a subject in need thereof the conjugate of any one of claims 1 to 43 or a pharmaceutical composition of any one of claims 44-46.

48. A method of eliciting targeted immune stimulation in a subject with a mesothelin-expressing cancer, comprising administering to a subject in need thereof the conjugate of any one of claims 1 to 43 or a pharmaceutical composition of any one of claims 44-46.

49. The method of claim 47 or claim 48, wherein the administering is in a regimen that comprises administering at least two cycles of the conjugate or pharmaceutical composition to the subject and wherein the regimen results in a Tmax of the conjugate in the subject of greater than 4 hours following each administration of the conjugate or pharmaceutical composition.

50. The method of claim 49, wherein the regimen comprises at least two cycles of administration of the conjugate or pharmaceutical composition to the subject and a total dose of greater than 0.4 mg/kg of the conjugate per cycle.

51. The method of claim 49 or claim 50, wherein the regimen comprises a total dose of greater than 0.5 mg/kg of the conjugate per cycle.

52. The method of any one of claims 49 to 51, wherein the regimen comprises three or more administrations of the conjugate or pharmaceutical composition, wherein the Tmax of the conjugate is greater than 4 hours following each administration.

53. The method of any one of claims 49 to 52, wherein the regimen results in a Tmax greater than 6 hours, greater than 8 hours, greater than 10 hours, greater than 12 hours, or greater than 15 hours following each administration of the conjugate or pharmaceutical composition.

54. The method of any one of claims 49 to 53, wherein Tmax is reached at or prior to 72 hours following each administration of the conjugate or pharmaceutical composition.

55. The method of claim 54, wherein Tmax is reached at or prior to 48 hours following each administration, at or prior to 30 hours following each administration, or at or prior to 24 hours following each administration.

56. The method of any one of claims 49 to 55, wherein the total dose per cycle is administered as a single dose.

57. The method of any one of claims 49 to 55, wherein the total dose per cycle is administered as a split-dose.

58. The method of any one of claims 49 to 57, wherein the total dose of the conjugate or pharmaceutical composition administered per cycle of the regimen from 0.5 to 7.5 mg/kg.

59. The method of claim 58, wherein the total dose of the conjugate is from 0.5 to 5 mg/kg, from

0. 5 to 4 mg/kg, from 0.5 to 3.5 mg/kg or from 0.5 to 2 mg/kg.

60. The method of any one of claims 49 to 59, wherein each cycle of the effective regimen is one week, two weeks, three weeks, or four weeks.

61. The method of any one of claims 49 to 60, wherein at least two doses of the conjugate or pharmaceutical composition are administered more than 7 days apart or more than 10 days apart.

62. The method of any one of claims 49 to 61, wherein there is a rest between at least one cycle of administration.

63. The method of any one of claims 49 to 62, wherein the conjugate or pharmaceutical composition is administered in at least two cycles, each cycle comprising a period of two weeks, three weeks for four week and wherein the total first dose of the conjugate administered per cycle is from about 0.5 to about 7.5 mg/kg.

64. The method of any one of claims 47 to 63 wherein the conjugate or pharmaceutical composition is administered subcutaneously.

65. The method of claim 64, wherein the conjugate or pharmaceutical composition is administered subcutaneously at each administration.

66. The method of any one of claims 47 to 63, wherein the conjugate or pharmaceutical composition is administered intravenously by a slow infusion that results in a Tmax of the conjugate in the subject of greater than 4 hours following each administration of the conjugate.

67. The method of any one of claims 47 to 66, comprising further administering a B-cell depleting agent to the subject.

68. The method of claim 67, wherein the B-cell depleting agent is an antibody.

69. The method of claim 68, wherein the B-cell depleting agent is an anti-CD19 or anti-CD20 antibody.

70. The method of any one of claims 67 to 69, wherein the B-cell depleting agent is administered at the same time as or within 14 days, within 7 days, within 1 day or within 24, 12, 6, 4, 3, 2, or 1 hour of the first administration of the pharmaceutical composition.

71. The method of any one of claims 67 to 70, wherein B cells are depleted prior to administration of the pharmaceutical composition.

72. The method of any one of claims 47 to 71, comprising monitoring the subject for an anaphylaxis-like toxicity following administration of the pharmaceutical composition.

73. The method of any one of claims 47 to 72, wherein the conjugate or pharmaceutical composition is administered with an agent that mitigates an anaphylactic-like toxicity.

74. The method of claim 73, wherein the agent that mitigates an anaphylactic-like toxicity is selected from epinephrine, an antihistamine, a cortisone, and a beta-agonist.

75. The method of any one of claims 47 to 74, wherein the subject has a a mesothelin-expressing cancer and the mesothelin-expressing cancer is a malignant mesothelioma, pancreatic cancer, ovarian cancer, pancreatic cancer, lung cancer, breast cancer.

76. The method of any one of claims 47 to 75, wherein the subject is a human.

77. A humanized antibody that specifically binds human mesothelin, wherein the antibody comprises a heavy chain variable region comprising complementarity determining regions (CDRs) having the amino acid sequences of the heavy chain variable region CDRs set forth in SEQ ID NO:9 and a light chain variable region comprising CDRs having the amino acid sequences of the light chain variable region CDRs set forth in SEQ ID NO:10.

78. The humanized antibody of claim 77, wherein the CDR residues are identified according to Kabat.

79. The humanized antibody of claim 77, wherein the antibody comprises a heavy chain (HC) CDR1 comprising the amino acid sequence of SEQ ID NO: 16, a HC CDR2 comprising the amino acid sequence of SEQ ID NO: 17 or 18, a HC CDR3 comprising the amino acid sequence of SEQ ID NO: 19, a light chain (LC) CDR1 comprising the amino acid sequence of SEQ ID NO: 20, a LC CDR2 comprising the amino acid sequence of SEQ ID NO: 21, and a LC CDR3 comprising the amino acid sequence of SEQ ID NO: 22.

80. A humanized antibody that specifically binds human mesothelin, wherein the antibody comprises a heavy chain variable region comprising the amino acid sequence set forth in SEQ ID NO:1.

81. The humanized antibody of any one of claims 77 to 79, wherein the antibody comprises a heavy chain variable region comprising the amino acid sequence set forth in SEQ ID NO:1.

82. The humanized antibody of claim 81, wherein the antibody comprises a heavy chain variable region comprising the amino acid sequence set forth in SEQ ID NO:2.

83. The humanized antibody of claim 81, wherein the antibody comprises a heavy chain variable region comprising the amino acid sequence set forth in SEQ ID NO:3.

84. The humanized antibody of claim 81, wherein the antibody comprises a heavy chain variable region comprising the amino acid sequence set forth in SEQ ID NO:4.

85. The humanized antibody of claim 81, wherein the antibody comprises a heavy chain variable region comprising the amino acid sequence set forth in SEQ ID NO:5.

86. The humanized antibody of claim 81, wherein the antibody comprises a heavy chain variable region comprising the amino acid sequence set forth in SEQ ID NO:6.

87. The humanized antibody of claim 81, wherein the antibody comprises a heavy chain variable region comprising the amino acid sequence set forth in SEQ ID NO:7.

88. The humanized antibody of claim 81, wherein the antibody comprises a heavy chain variable region comprising the amino acid sequence set forth in SEQ ID NO:8.

89. The humanized antibody of claim 81, wherein the antibody comprises a heavy chain variable region comprising the amino acid sequence set forth in SEQ ID NO:9.

90. The humanized antibody of any one of claims 77 to 89, wherein the antibody comprises a light chain variable region comprising the amino acid sequence set forth in SEQ ID NO:10.

91. The humanized antibody of any one of claims 77 to 89, wherein the antibody comprises a light chain variable region comprising the amino acid sequence set forth in SEQ ID NO:11.

92. The humanized antibody of any one of claims 77 to 89, wherein the antibody comprises a light chain variable region comprising the amino acid sequence set forth in SEQ ID NO:12.

93. The humanized antibody of any one of claims 77 to 89, wherein the antibody comprises a light chain variable region comprising the amino acid sequence set forth in SEQ ID NO:13.

94. The humanized antibody of any one of claims 77 to 89, wherein the antibody comprises a light chain variable region comprising the amino acid sequence set forth in SEQ ID NO:14.

95. The humanized antibody of any one of claims 77 to 89, wherein the antibody comprises a light chain variable region comprising the amino acid sequence set forth in SEQ ID NO:15.

96. The humanized antibody of any one of claims 77 to 79, wherein the antibody comprises a heavy chain variable region comprising the amino acid sequence set forth in SEQ ID NO:1 and a light chain variable region comprising the amino acid sequence set forth in SEQ ID NO:10 or SEQ ID NO:15.

97. The humanized antibody of claim 96, wherein the antibody comprises a heavy chain variable region comprising the amino acid sequence set forth in SEQ ID NO:8 and a light chain variable region comprising the amino acid sequence set forth in SEQ ID NO:11.

98. The humanized antibody of claim 96, wherein the antibody comprises a heavy chain variable region comprising the amino acid sequence set forth in SEQ ID NO:9 and a light chain variable region comprising the amino acid sequence set forth in SEQ ID NO:11.

99. The humanized antibody of claim 96, wherein the antibody comprises a heavy chain variable region comprising the amino acid sequence set forth in SEQ ID NO:9 and a light chain variable region comprising the amino acid sequence set forth in SEQ ID NO:15.

100. The humanized antibody of any one of claims 77 to 79, wherein the antibody comprises a heavy chain variable region comprising the amino acid sequence set forth in SEQ ID NO:25 or SEQ ID NO:27 and a light chain variable region comprising the amino acid sequence set forth in SEQ ID NO:26

101. The humanized antibody of any one of claims 77 to 100, wherein the antibody is an IgG1 antibody.

102. The humanized antibody of any one of claims 77 to 101, wherein the antibody comprises a heavy chain constant region comprising the amino acid sequence set forth in SEQ ID NO:23 and a light chain constant region comprising the amino acid sequence set forth in SEQ ID NO:24.

103. The humanized antibody or of any one of claims 77 to 101, wherein the antibody comprises a wild-type IgG1 Fc domain or an IgG1 Fc domain variant having the same or substantially similar binding affinity to FcγRI, FcγRII, and FcγRIII as compared to a wild-type IgG1 Fc domain.

104. The humanized antibody of any one of claims 77 to 101, wherein the antibody comprises a wild-type IgG1 Fc domain or an IgG1 Fc domain variant having the same or substantially similar binding affinity to FcRn as compared to a wild-type IgG1 Fc domain.

105. The humanized antibody of any one of claims 77 to 101, wherein the antibody comprises a wild-type IgG1 Fc domain or an IgG1 Fc domain variant having increased and/or decreased affinity to one or more Fcγ receptors as compared to a wild-type IgG1 Fc domain.

106. The humanized antibody of any one of claims 77 to 105, wherein the antibody is a full-length antibody.

107. The humanized antibody of any one of claims 77 to 105, wherein the antibody is an antigen binding fragment.

108. A conjugate comprising the humanized antibody of any one of claims 77 to 107 conjugated to an immune-stimulatory compound.

109. A conjugate comprising the humanized antibody of any one of claims 77 to 107 conjugated to a cytotoxic compound.

110. The conjugate of claim 108 wherein the immune-stimulatory compound is a benzazepine drug.

111. An isolated nucleic acid encoding the humanized antibody of any one of claims 77 to

107.

112. An expression vector comprising the isolated nucleic acid of claim 111.

113. A host cell comprising the isolated nucleic acid of claim 111 or the expression vector of claim 112.

114. A host cell that expresses the humanized antibody of any one of claims 77 to 107.

115. A method of producing a humanized antibody comprising culturing the host cell of claim 112 or claim 114 under conditions suitable for expressing the humanized antibody.

116. The method of claim 115, further comprising isolating the humanized antibody.
